(19) United States
(12) Patent Application Publication
(10) Pub. No.: US 2023/0212684 A1
(43) Pub. Date: Jul. 6, 2023

(54) CELL-FREE DNA BIOMARKERS AND THEIR USE IN DIAGNOSIS, MONITORING RESPONSE TO THERAPY, AND SELECTION OF THERAPY FOR PROSTATE CANCER

(71) Applicant: The Board of Trustees of the Leland Stanford Junior University, Stanford, CA (US)

(72) Inventors: Shan X. WANG, Stanford, CA (US); Ritish PATNAIK, Stanford, CA (US)

(73) Assignee: The Board of Trustees of the Leland Stanford Junior University, Stanford, CA (US)

(21) Appl. No.: 17/923,549

(22) PCT Filed: May 4, 2021
(86) PCT No.: PCT/US21/30684

§ 371 Date: Nov. 4, 2022

Related U.S. Application Data
(60) Provisional application No. 63/020,266, filed on May 5, 2020.

(51) Int. Cl. C12Q 1/6886; C12Q 1/6806

(52) U.S. Cl. CPC C12Q 1/6886; C12Q 1/6806; C12Q 2600/106; C12Q 2600/154

(57) ABSTRACT

Compositions, methods, and kits are provided for diagnosing and treating prostate cancer. In particular, cell-free DNA biomarkers have been identified that can be used to aid in diagnosis, selection of treatment, and monitoring of treatment of prostate cancer.

### BACKGROUND

Prostate cancer (PRAD) is the most common cancer diagnosed in men in the United States, with ˜192,000 estimated new cases in 2020 (Xu et al. (2017) Nature Materials 16:1155-1161; Luo et al. (2020) Science Translational Medicine 12(524) eaax7533; Litwin et al. (2017) JAMA 317(24):2532-2542; American Cancer Society. Cancer Facts & FIGS. 2020. Atlanta: American Cancer Society; 2020). While the potential adverse events from therapy and the typically indolent effect of tumors have called into question the importance of early treatment, prostate cancer remains the third leading cause of cancer death in U.S. men, especially those with metastatic prostate cancer. Metastatic prostate cancer patients first undergo androgen deprivation therapy (ADT), but unfortunately, many patients become resistant to androgen therapy, resulting in palliative therapy for these patients who are now considered to have castration-resistant prostate cancer (CRPC). For palliative therapy, a combination of chemotherapy with docetaxel and the androgen inhibitor, abiraterone acetate (AA), is often used, as this has been shown to boost overall survival in contrast to treatment with abiraterone acetate after docetaxel (Komura et al. (2018) Int J Urol. 25(3):220-231).

There remains a need for better methods of diagnosing and treating prostate cancer.

### SUMMARY

Compositions, methods, and kits are provided for diagnosing and treating prostate cancer. In particular, cell-free DNA biomarkers have been identified that can be used to aid in diagnosis, selection of treatment, and monitoring of treatment of prostate cancer.

In one aspect, a method of diagnosing and treating prostate cancer in a patient is provided, the method comprising: a) obtaining a blood sample from the patient; b) measuring frequency of methylation at one or more CpG sites in at least one biomarker gene selected from the group consisting of APC, CD44, PYCARD, RARB, and RBP1 in cell-free DNA (cfDNA) in the blood sample, wherein increased frequency of methylation at the one or more CpG sites compared to reference value ranges for the frequency of methylation at the one or more CpG sites in said at least one biomarker gene selected from the group consisting of APC, CD44, PYCARD, RARB, and RBP1 in cfDNA from a control subject indicate that the patient has prostate cancer; and c) treating the patient for the prostate cancer, if the patient has a positive diagnosis for prostate cancer based on the frequency of methylation at the one or more CpG sites.

In certain embodiments, the one or more CpG sites are selected from cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124 (locations of these CpG sites are given in the Illumina HumanMethylation450K Manifest), and CpG sites located within 200 nucleotides thereof. In some embodiments, the frequency of methylation is measured at all the cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124 CpG sites in the cfDNA.

In certain embodiments, the method further comprising calculating a prostate cancer risk score based on the methylation frequency at the CpG sites in the APC, CD44, PYCARD, RARB, and RBP1 genes of the cfDNA using one or more algorithms.

Exemplary methods for treating a patient for prostate cancer include, without limitation, performing a surgical resection, a prostatectomy, external beam radiation therapy, particle therapy, brachytherapy, cryosurgery, or high-intensity focused ultrasound, or administering androgen deprivation therapy, chemotherapy, targeted therapy, or immunotherapy, or a combination thereof.

In certain embodiments, the method further comprises measuring blood levels of prostate-specific antigen (PSA), blood levels of kallikrein 2, or urine levels of prostate cancer antigen 3 (PCA3), or a combination thereof, wherein detection of increased blood levels of PSA, increased blood levels of kallikrein 2, or increased urine levels of PCA3 in combination with increased frequency of methylation at the one or more CpG sites in the one or more genes selected from the group consisting of APC, CD44, PYCARD, RARB, and RBP1 compared to reference value ranges for the blood levels of PSA and kallikrein 2, the urine levels of PCA3, and the frequency of methylation at the one or more CpG sites in the one or more genes selected from the group consisting of APC, CD44, PYCARD, RARB, and RBP1 for a control subject indicate that the patient has a positive diagnosis for the prostate cancer.

In certain embodiments, the patient has a risk factor that makes the patient susceptible to developing prostate cancer. Risk factors include, without limitation, an age over 50, a family history including a family member or close relative who has had prostate cancer, a genetic predisposition to develop prostate cancer, an environmental exposure to a carcinogen, or a disease or condition that increases the risk of developing prostate cancer such as obesity, a sexually transmitted disease, or prostatitis.

In another aspect, a method of monitoring prostate cancer in a patient is provided, the method comprising: a) obtaining a first blood sample from the patient at a first time point and a second blood sample from the patient later at a second time point; and b) detecting methylation at one or more CpG sites in one or more genes of circulating free DNA (cfDNA) in the first blood sample and the second blood sample, wherein the one or more genes are selected from the group consisting of APC, CD44, PYCARD, RARB, and RBP1, wherein detection of increased frequency of methylation of the CpG sites in the one or more genes selected from the group consisting of APC, CD44, PYCARD, RARB, and RBP1 in the cfDNA of the second blood sample compared to the cfDNA of the first blood sample indicate that the prostate cancer is progressing, and detection of decreased frequency of methylation of the CpG sites in the one or more genes selected from the group consisting of APC, CD44, PYCARD, RARB, and RBP1 in the cfDNA of the second blood sample compared to the cfDNA of the first blood sample indicate that the prostate cancer is not progressing. In some embodiments, the first time point is before a treatment of the patient for prostate cancer is started and the second time point is during or after the treatment.

In certain embodiments, the prostate cancer is a primary tumor, a metastasis, or a recurrence.

In certain embodiments, the treatment comprises performing a surgical resection, a prostatectomy, external beam radiation therapy, particle therapy, brachytherapy, cryosurgery, or high-intensity focused ultrasound, or administering androgen deprivation therapy, chemotherapy, targeted therapy, or immunotherapy, or a combination thereof.

In certain embodiments, the method further comprises repeating steps a) and b).

In certain embodiments, the method further comprises increasing dosage or frequency of a treatment for prostate cancer, changing to a different treatment, or starting palliative care for the patient if the prostate cancer is progressing.

In certain embodiments, the one or more CpG sites are selected from cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124 (locations of these CpG sites are given in the Illumina HumanMethylation450K Manifest), and CpG sites located within 200 nucleotides thereof. In some embodiments, the frequency of methylation is measured at all the cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124 CpG sites in the cfDNA.

In certain embodiments, the method further comprises measuring blood levels of prostate-specific antigen (PSA), blood levels of kallikrein 2, or urine levels of prostate cancer antigen 3 (PCA3), or a combination thereof, wherein detection of increased blood levels of PSA, increased blood levels of kallikrein 2, or increased urine levels of PCA3 in combination with increased frequency of methylation at the one or more CpG sites in the one or more genes selected from the group consisting of APC, CD44, PYCARD, RARB, and RBP1 in the second blood sample compared to the first blood sample indicate that the prostate cancer is progressing; and decreased blood levels of prostate-specific antigen (PSA), decreased blood levels of kallikrein 2, or decreased urine levels of prostate cancer antigen 3 (PCA3) in combination with decreased frequency of methylation at the one or more CpG sites in the one or more genes selected from the group consisting of APC, CD44, PYCARD, RARB, and RBP1 in the second blood sample compared to the first blood sample indicate that the prostate cancer is not progressing.

In another aspect, a method of monitoring for a recurrence of prostate cancer in a patient is provided, the method comprising: a) obtaining a first circulating free DNA (cfDNA) sample from the patient after treatment for a previous occurrence of prostate cancer at a first time point when the patient is characterized as cancer-free from imaging or other diagnostic modalities; b) detecting methylation at one or more CpG sites of one or more biomarker genes in cfDNA from the first cfDNA sample, wherein the one or more biomarker genes are selected from APC, CD44, PYCARD, RARB, and RBP1; c) obtaining a second cfDNA sample from the patient at a second time point during a period of monitoring for the recurrence; d) detecting methylation at the one or more CpG sites of the one or more biomarker genes in cfDNA from the second cfDNA sample, wherein the one or more biomarker genes are selected from APC, CD44, PYCARD, RARB, and RBP1, wherein increased frequency of methylation at the one or more CpG sites of the one or more biomarker genes selected from APC, CD44, PYCARD, RARB, and RBP1 in the cfDNA of the second cfDNA sample compared to the cfDNA of the first cfDNA sample indicates that the prostate cancer has recurred; and e) repeating steps c)-e) subsequently during the period of monitoring for the recurrence.

In certain embodiments, the method further comprises treating the patient for the recurrence of the prostate cancer, if the patient has a positive diagnosis for the recurrence of the prostate cancer based on the levels of methylation of the one or more CpG sites.

In certain embodiments, the patient is treated for the recurrence of prostate cancer by performing a surgical resection, prostatectomy, external beam radiation therapy, particle therapy, brachytherapy, cryosurgery, or high-intensity focused ultrasound, or administering androgen deprivation therapy, chemotherapy, targeted therapy, or immunotherapy, or a combination thereof.

In certain embodiments, the one or more CpG sites are selected from cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124 (locations of these CpG sites are given in the Illumina HumanMethylation450K Manifest), and CpG sites located within 200 nucleotides thereof. In some embodiments, the frequency of methylation is measured at all the cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124 CpG sites in the cfDNA.

In certain embodiments, the method further comprises measuring blood levels of prostate-specific antigen (PSA), blood levels of kallikrein 2, or urine levels of prostate cancer antigen 3 (PCA3), or a combination thereof for the patient, wherein increased blood levels of PSA, increased blood levels of kallikrein 2, or increased urine levels of PCA3 in combination with increased frequency of methylation at the one or more CpG sites in the one or more genes selected from the group consisting APC, CD44, PYCARD, RARB, and RBP1 in the cfDNA from the patient compared to reference value ranges for the blood levels of PSA, the blood levels of kallikrein 2, the urine levels of PCA3, and the frequency of methylation at the one or more CpG sites in the one or more genes selected from the group consisting of APC, CD44, PYCARD, RARB, and RBP1 indicate that the patient has a positive diagnosis for the recurrence of prostate cancer.

In certain embodiments, the cfDNA sample is a blood sample or plasma sample comprising cfDNA.

In another aspect, a method for selecting an individual with prostate cancer for treatment with abiraterone acetate (17-(3-pyridinyl)androsta-5,16-dien-3β-ol acetate) and treating the individual is provided, the method comprising: a) obtaining a blood sample from the individual; b) measuring frequency of methylation at one or more CpG sites in at least one biomarker gene selected from the group consisting of RARB and RBP1 in cell-free DNA (cfDNA) in the blood sample, wherein increased frequency of methylation at the one or more CpG sites compared to reference value ranges for the frequency of methylation at the one or more CpG sites in said at least one biomarker gene selected from the group consisting of RARB and RBP1 in cfDNA from a control subject indicate that the individual will benefit from treatment with the abiraterone acetate; and c) administering a therapeutically effective amount of the abiraterone acetate to the individual, if the frequency of methylation at the one or more CpG sites in said at least one biomarker gene selected from the group consisting of RARB and RBP1 indicates that the individual will benefit from treatment with the abiraterone acetate.

In certain embodiments, the one or more CpG sites are selected from cg02499249, cg26124016, cg12479047, cg20899354, cg18094781, cg06720425, and cg15229124. In some embodiments, the frequency of methylation is measured at all the cg02499249, cg26124016, cg12479047, cg20899354, cg18094781, cg06720425, and cg15229124 CpG sites in the cfDNA.

In certain embodiments, the individual is treated with the abiraterone acetate in combination with docetaxel and/or a corticosteroid.

In the practice of the subject methods, methylation at the CpG sites in the cfDNA can be detected using any method known in the art for detecting methylation of DNA, including, without limitation, methylation-sensitive arbitrarily-primed polymerase chain reaction (MS AP-PCR), methylation-sensitive single nucleotide primer extension (Ms-SNuPE), methylation-specific PCR (MSP), methylation-sensitive DNA restriction enzyme analysis, restriction enzyme-based sequencing, restriction enzyme-based microarray analysis, combined bisulfite restriction analysis (COBRA), methylated CpG island amplification (MCA), methylated CpG island amplification and microarray (MCAM), HpaII tiny fragment enrichment by ligation-mediated PCR (HELP), bisulfite sequencing, bisulfite microarray analysis, methylation-specific pyrosequencing, HELP-sequencing (HELP-seq), TET-assisted pyridine borane sequencing (TAPS), GlaI hydrolysis and ligation adapter dependent PCR (GLAD-PCR), methylated DNA immunoprecipitation-sequencing (MeDIP-Seq), or methylated DNA immunoprecipitation-microarray analysis (MeDI P-chip), Southern blotting with methyl-sensitive restriction enzymes, or methylation-specific giant magnetoresistive sensor-based microarray analysis.

In another aspect, a kit comprising agents for detecting methylation of one or more CpG sites in APC, CD44, PYCARD, RARB, and RBP1 genes in cfDNA is provided.

In certain embodiments, the kit comprises agents for detecting methylation at one or more CpG sites selected from cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124, and CpG sites located within 200 nucleotides thereof. In some embodiments, the kit comprises agents for detecting methylation at all the cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124 CpG sites in the cfDNA.

In certain embodiments, the kit further comprises agents for performing methylation-sensitive arbitrarily-primed polymerase chain reaction (MS AP-PCR), methylation-sensitive single nucleotide primer extension (Ms-SNuPE), methylation-specific PCR (MSP), methylation-sensitive DNA restriction enzyme analysis, restriction enzyme-based sequencing, restriction enzyme-based microarray analysis, combined bisulfite restriction analysis (COBRA), methylated CpG island amplification (MCA), methylated CpG island amplification and microarray (MCAM), HpaII tiny fragment enrichment by ligation-mediated PCR (HELP), bisulfite sequencing, bisulfite microarray analysis, methylation-specific pyrosequencing, HELP-sequencing (HELP-seq), TET-assisted pyridine borane sequencing (TAPS), GlaI hydrolysis and ligation adapter dependent PCR (GLAD-PCR), methylated DNA immunoprecipitation-sequencing (MeDIP-Seq), or methylated DNA immunoprecipitation-microarray analysis (MeDI P-chip), Southern blotting with methyl-sensitive restriction enzymes, or methylation-specific giant magnetoresistive sensor-based microarray analysis. In some embodiments, the agents comprise a bisulfite reagent, methylation-sensitive restriction enzymes, PCR primers that selectively amplify DNA regions that contain CpG dinucleotides, methylation-specific primers, methylation-specific probes, or a combination thereof.

In certain embodiments, the kit further comprises reagents for measuring PSA, kallikrein 2, or PCA3, or a combination thereof.

In certain embodiments, the kit further comprises instructions for using the kit for diagnosis of prostate cancer, detecting recurrence of prostate cancer, or monitoring treatment of prostate cancer.

In another aspect, a cell-free DNA hypermethylated at one or more CpG sites in at least one biomarker gene selected from APC, CD44, PYCARD, RARB, and RBP1 is provided for use in diagnosing prostate cancer, detecting recurrence of prostate cancer, or monitoring treatment of prostate cancer.

In certain embodiments, one or more CpG sites are selected from cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124, and CpG sites located within 200 nucleotides thereof.

In another aspect, an in vitro method of diagnosing prostate cancer in a patient is provided, the method comprising: a) obtaining a blood sample from the patient; and b) measuring frequency of methylation at one or more CpG sites in at least one biomarker gene selected from the group consisting of APC, CD44, PYCARD, RARB, and RBP1 in cell-free DNA (cfDNA) in the blood sample, wherein increased frequency of methylation at the one or more CpG sites compared to reference value ranges for the frequency of methylation at the one or more CpG sites in said at least one biomarker gene selected from the group consisting of APC, CD44, PYCARD, RARB, and RBP1 in cfDNA from a control subject indicate that the patient has prostate cancer.

In certain embodiments, one or more CpG sites are selected from cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124, and CpG sites located within 200 nucleotides thereof. In some embodiments, the frequency of methylation is measured at all the cg02499249, cg26124016, cg12479047, cg20899354, cg18094781, cg06720425, and cg15229124 CpG sites in the cfDNA.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. Layered Analysis for Methylated Biomarkers for Prostate cancer.

FIG. 2. LAMB-PRAD Score Calculation for Each Visit through Geometric Mean.

FIG. 3. Box-and-whisker plot and time-course plot of LAMB-PRAD scores of CRPC patients for therapy response.

FIG. 4. Representative non-responder LAMB-PRAD score plots for therapy monitoring.

FIG. 5. Time-course plot of LAMB-PRAD scores for a non-responder patient (PMH118) who later showed response to therapy (dotted line: visit with response).

FIG. 6. Time-course plots of LAMB-PRAD scores for responder patients who later showed resistance to therapy (dotted line: visit with resistance).

FIG. 7. Box-and-whisker plot of RARB/RBP1 baseline scores for therapy selection.

FIG. 8. Flow Diagram for Selecting Tissue Studies for Meta-Analysis.

### DETAILED DESCRIPTION OF EMBODIMENTS

Compositions, methods, and kits are provided for diagnosing and treating prostate cancer. In particular, methylated cell-free DNA biomarkers have been identified that can be used to aid in diagnosis, selection of treatment, and monitoring of treatment of prostate cancer.

Before the methylated cell-free DNA biomarkers and methods of using them are described, it is to be understood that this invention is not limited to a particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cfDNA” includes a plurality of such cfDNAs and reference to “the cancerous cell” includes reference to one or more cancerous cells and equivalents thereof, such as cancer cells, tumor cells, neoplastic cells, and malignant cells, known to those skilled in the art, and so forth.

As used herein, the term “circulating cell-free DNA” refers to DNA that is circulating in the peripheral blood of a patient. The DNA molecules in cell-free DNA may have a median size that is below 1 kb (e.g., in the range of 50 bp to 500 bp, 80 bp to 400 bp, or 100-1,000 bp), although fragments having a median size outside of this range may be present. Cell-free DNA may contain circulating tumor DNA (ctDNA), i.e., tumor DNA circulating freely in the blood of a cancer patient or circulating fetal DNA (if the subject is a pregnant female). cfDNA can be highly fragmented and in some cases can have a mean fragment size about 165-250 bp (Newman et al Nat Med. 2014 20: 548-54). cfDNA can be obtained by centrifuging whole blood to remove all cells, and then isolating the DNA from the remaining plasma or serum. Such methods are well known (see, e.g., Lo et al, Am J Hum Genet 1998; 62:768-75). Circulating cell-free DNA is double-stranded, but can be made single stranded by denaturation.

By “isolated” is meant, when referring to a polypeptide, that the indicated molecule is separate and discrete from the whole organism with which the molecule is found in nature or is present in the substantial absence of other biological macro molecules of the same type. The term “isolated” with respect to a nucleic acid (e.g., cell-free DNA) is a nucleic acid molecule devoid, in whole or part, of sequences normally associated with it in nature; or a sequence, as it exists in nature, but having heterologous sequences in association therewith; or a molecule disassociated from the chromosome.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with cancer) as well as those in which prevention is desired (e.g., those with increased susceptibility to cancer, those suspected of having cancer, those with a risk of recurrence, etc.).

The terms “tumor,” “cancer” and “neoplasia” are used interchangeably and refer to a cell or population of cells whose growth, proliferation or survival is greater than growth, proliferation or survival of a normal counterpart cell, e.g. a cell proliferative, hyperproliferative or differentiative disorder. Typically, the growth is uncontrolled. The term “malignancy” refers to invasion of nearby tissue. The term “metastasis” or a secondary, recurring or recurrent tumor, cancer or neoplasia refers to spread or dissemination of a tumor, cancer or neoplasia to other sites, locations or regions within the subject, in which the sites, locations or regions are distinct from the primary tumor or cancer. Neoplasia, tumors and cancers include benign, malignant, metastatic and non-metastatic types, and include any stage (I, II, III, IV or V) or grade (G1, G2, G3, etc.) of neoplasia, tumor, or cancer, or a neoplasia, tumor, cancer or metastasis that is progressing, worsening, stabilized or in remission. In particular, the terms “tumor,” “cancer” and “neoplasia” include carcinomas, such as squamous cell carcinoma, adenocarcinoma, adenosquamous carcinoma, anaplastic carcinoma, large cell carcinoma, and small cell carcinoma.

“Prostate cancer” refers to any type of prostate cancer, including, without limitation, prostate adenocarcinoma (e.g., acinar, ductal, or intraductal adenocarcinoma), atrophic carcinoma, microcystic carcinoma, foamy gland carcinoma, small cell prostate cancer, non-small cell prostate cancer, neuroendocrine prostate cancer, and metastatic castration resistant prostate cancer. The prostate cancer may be of any type, stage or grade, and may have, for example, a Gleason score of 2 to 10.

An “androgen-responsive cancer” includes any cancer that is responsive to androgenic hormones that bind to the androgen receptor, including testosterone and dihydrotestosterone, which promote cancer growth. In certain embodiments, the androgen-responsive cancer is prostate cancer such as, but not limited to, prostate adenocarcinoma and includes other types of cancers (e.g., neuroendocrine, acinar, or ductal adenocarcinoma), transitional cell cancer, sarcoma, carcinoid, or small cell carcinoma. The prostate cancer may be of any type, stage or grade, and may have, for example, a Gleason score of 2 to 10. In certain embodiments, the prostate cancer is CRPC or castrate (or hormone)-refractory prostate cancer (i.e., prostate cancer resistant to androgen deprivation therapy).

By “resistant to androgen deprivation therapy” is meant, when referring to a patient that has prostate cancer, that the condition of the patient does not improve with androgen deprivation therapy. Patients who are resistant to androgen deprivation therapy may experience tumor growth or cancer metastasis after androgen deprivation therapy treatment and reduced survival time compared to patients who respond to androgen deprivation therapy.

The term “survival” as used herein means the time from the start of treatment to the time of death.

By “anti-tumor activity” is intended a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Such activity can be assessed using animal models.

The term “tumor response” as used herein means a reduction or elimination of all measurable lesions. The criteria for tumor response are based on the WHO Reporting Criteria [WHO Offset Publication, 48-World Health Organization, Geneva, Switzerland, (1979)]. Ideally, all uni- or bidimensionally measurable lesions should be measured at each assessment. When multiple lesions are present in any organ, such measurements may not be possible and, under such circumstances, up to 6 representative lesions should be selected, if available.

The term “complete response” (CR) as used herein means a complete disappearance of all clinically detectable malignant disease, determined by 2 assessments at least 4 weeks apart.

The term “partial response” (PR) as used herein means a 50% or greater reduction from baseline in the sum of the products of the longest perpendicular diameters of all measurable disease without progression of evaluable disease and without evidence of any new lesions as determined by at least two consecutive assessments at least four weeks apart. Assessments should show a partial decrease in the size of lytic lesions, recalcifications of lytic lesions, or decreased density of blastic lesions.

“Substantially purified” generally refers to isolation of a substance (compound, drug, polynucleotide, protein, polypeptide) such that the substance comprises the majority percent of the sample in which it resides. Typically in a sample, a substantially purified component comprises 50%, preferably 80%-85%, more preferably 90-95% of the sample. Techniques for purifying substances of interest are well-known in the art and include, for example, ion-exchange chromatography, affinity chromatography and sedimentation according to density.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

As used herein, the terms “detectable label”, “detection agent”, “diagnostic agent”, and “detectable moiety” are used interchangeably and refer to a molecule or substance capable of detection, including, but not limited to, fluorescers, chemiluminescers, chromophores, bioluminescent proteins, enzymes, enzyme substrates, enzyme cofactors, enzyme inhibitors, isotopic labels, semiconductor nanoparticles, dyes, metal ions, metal sols, ligands (e.g., biotin, streptavidin or haptens) and the like. The term “fluorescer” refers to a substance or a portion thereof which is capable of exhibiting fluorescence in the detectable range. Particular examples of detectable labels which may be used in the practice of the invention include isotopic labels, including radioactive (e.g., alpha, beta, gamma, or positron-emitting radionuclides) and non-radioactive isotopes, such as, ³H, ²H, ¹²⁰I, ¹²³I, ¹²⁴I, ¹²⁵I, ¹³¹I, ³⁵S, ¹¹C, ¹³C, ¹⁴C, ³²P, ¹⁵N, ¹³N, ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹⁵⁴Gd, ¹⁵⁵Gd, ¹⁵⁶Gd, ¹⁵⁷Gd, ¹⁵⁸Gd, ¹⁵O, ¹⁸⁶Re, ¹⁸⁸Re, ⁵¹M, ^{52m}Mn, ⁵⁵Co, ⁷²As, ⁷⁵Br, ⁷⁸Br, ^{82m}Rb, and ⁸³Sr. In particular, detectable labels may comprise positron-emitting radionuclides suitable for PET imaging such as, but not limited to, ⁶⁴Cu, ⁸⁹Zr, ⁶⁸Ga, ¹⁷⁷Lu, ⁸²Rb, ¹¹C, ¹³N, ¹⁵O, and ¹⁸F; gamma-emitting radionuclides suitable for single photon emission computed tomography (SPECT) imaging such as, but not limited to, ⁶⁷Ga, ^{99m}Tc, ¹²³I, and ¹³¹I; and radionuclides suitable for radiotherapy such as ¹⁷⁷Lu, ⁹⁰Y, ²¹³Bi, ²¹²Pb, ²¹¹At, ²²⁵Ac, ¹⁶⁶Ho, ⁸⁹Sr, ¹⁵³Sm, ²²³Ra, ²²⁶Ra, ¹³⁷Cs, ¹⁹⁸Au, ¹⁸²Ta, ¹⁹²Ir, ¹²⁵I, or ¹³¹I. Detectable labels may also include non-radioactive, paramagnetic metal ions suitable for MRI imaging such as, but not limited to, Mn²⁺, Fe³⁺, Fe²⁺, Gd³⁺, Ti²⁺, Cr³⁺, Co²⁺, Ni²⁺, and Cu²⁺. Detectable labels may also include fluorophores including without limitation, SYBR green, SYBR gold, a CAL Fluor dye such as CAL Fluor Gold 540, CAL Fluor Orange 560, CAL Fluor Red 590, CAL Fluor Red 610, and CAL Fluor Red 635, a Quasar dye such as Quasar 570, Quasar 670, and Quasar 705, an Alexa Fluor such as Alexa Fluor 350, Alexa Fluor 488, Alexa Fluor 546, Alexa Fluor 555, Alexa Fluor 594, Alexa Fluor 647, and Alexa Fluor 784, a cyanine dye such as Cy 3, Cy3.5, Cy5, Cy5.5, and Cy7, fluorescein, 2′, 4′, 5′, 7′-tetrachloro-4-7-dichlorofluorescein (TET), carboxyfluorescein (FAM), 6-carboxy-4′,5′-dichloro-2′,7′-dimethoxyfluorescein (JOE), hexachlorofluorescein (HEX), rhodamine, carboxy-X-rhodamine (ROX), tetramethyl rhodamine (TAMRA), FITC, dansyl, umbelliferone, dimethyl acridinium ester (DMAE), Texas red, luminol, and quantum dots, enzymes such as alkaline phosphatase (AP), beta-lactamase, chloramphenicol acetyltransferase (CAT), adenosine deaminase (ADA), aminoglycoside phosphotransferase (neo^{r}, G418^{r}) dihydrofolate reductase (DHFR), hygromycin-B-phosphotransferase (HPH), thymidine kinase (TK), β-galactosidase (lacZ), and xanthine guanine phosphoribosyltransferase (XGPRT), beta-glucuronidase (gus), placental alkaline phosphatase (PLAP), and secreted embryonic alkaline phosphatase (SEAP). Enzyme tags are used with their cognate substrate. The terms also include chemiluminescent labels such as luminol, isoluminol, acridinium esters, and peroxyoxalate and bioluminescent proteins such as firefly luciferase, bacterial luciferase, Renilla luciferase, and aequorin. The terms also include color-coded microspheres of known fluorescent light intensities (see e.g., microspheres with xMAP technology produced by Luminex (Austin, Tex.); microspheres containing quantum dot nanocrystals, for example, containing different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), glass microparticles with digital holographic code images (see e.g., CyVera microbeads produced by Illumina (San Diego, Calif.), near infrared (NIR) probes, and nanoshells. The terms also include contrast agents such as ultrasound contrast agents (e.g. SonoVue microbubbles comprising sulfur hexafluoride, Optison microbubbles comprising an albumin shell and octafluoropropane gas core, Levovist microbubbles comprising a lipid/galactose shell and an air core, Perflexane lipid microspheres comprising perfluorocarbon microbubbles, and Perflutren lipid microspheres comprising octafluoropropane encapsulated in an outer lipid shell), magnetic resonance imaging (MRI) contrast agents (e.g., gadodiamide, gadobenic acid, gadopentetic acid, gadoteridol, gadofosveset, gadoversetamide, gadoxetic acid), and radiocontrast agents, such as for computed tomography (CT), radiography, or fluoroscopy (e.g., diatrizoic acid, metrizoic acid, iodamide, iotalamic acid, ioxitalamic acid, ioglicic acid, acetrizoic acid, iocarmic acid, methiodal, diodone, metrizamide, iohexol, ioxaglic acid, iopamidol, iopromide, iotrolan, ioversol, iopentol, iodixanol, iomeprol, iobitridol, ioxilan, iodoxamic acid, iotroxic acid, ioglycamic acid, adipiodone, iobenzamic acid, iopanoic acid, iocetamic acid, sodium iopodate, tyropanoic acid, and calcium iopodate).

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium). The term “antibody” encompasses monoclonal antibodies as well as hybrid antibodies, altered antibodies, chimeric antibodies, and humanized antibodies.

The term antibody includes: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (scFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

Biomarkers. The term “biomarker” as used herein refers to a compound, such as methylated cell-free DNA, a protein, a mRNA, a metabolite, or a metabolic byproduct which is differentially expressed or present at different concentrations, levels or frequencies in one sample compared to another, such as a sample of hypermethylated cell-free DNA derived from patients who have prostate cancer compared to cell-free DNA derived from healthy control subjects (i.e., subjects not having cancer). Biomarkers include, but are not limited to, cfDNA methylated at one or more CpG sites in one or more biomarker genes selected from APC, CD44, PYCARD, RARB, and RBP1 having increased frequency of methylation in cell-free DNA from patients who have prostate cancer compared to cell-free DNA derived from healthy control subjects. Biomarkers include cfDNA with an increased frequency or level of methylation at one or more CpG sited selected from cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124, and CpG sites located within 200 nucleotides thereof (methylated CpG sites for each biomarker gene are listed in Table 3). In some embodiments, the biomarkers include cfDNA with differentially methylated CpGs in the promoters of tumor suppressors.

In some embodiments, the concentration or level of a biomarker is determined before and after the administration of an anti-cancer treatment to a subject. The degree of change in the frequency or level of a biomarker (e.g., methylation of CpG motifs in APC, CD44, PYCARD, RARB, or RBP1), or lack thereof, is interpreted as an indication of whether the anti-cancer treatment has the desired effect (e.g., anti-tumor activity). In other words, the frequency or level of a biomarker is determined before and after the administration of the anti-cancer treatment to an individual, and the degree of change, or lack thereof, in the level is interpreted as an indication of whether the individual is “responsive” to the treatment.

A “reference level” or “reference value” of a biomarker means a level of the biomarker that is indicative of a particular disease state, phenotype, or predisposition to developing a particular disease state or phenotype, or lack thereof, as well as combinations of disease states, phenotypes, or predisposition to developing a particular disease state or phenotype, or lack thereof. A “positive” reference level of a biomarker means a level that is indicative of a particular disease state or phenotype. A “negative” reference level of a biomarker means a level that is indicative of a lack of a particular disease state or phenotype. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched or gender-matched so that comparisons may be made between biomarker levels in samples from subjects of a certain age or gender and reference levels for a particular disease state, phenotype, or lack thereof in a certain age or gender group). Such reference levels may also be tailored to specific techniques that are used to measure levels of biomarkers in biological samples (e.g., PCR, microarray analysis, mass spectrometry (e.g., LC-MS, GC-MS), tandem mass spectrometry, NMR, biochemical or enzymatic assays, etc.), where the levels of biomarkers may differ based on the specific technique that is used.

A “similarity value” is a number that represents the degree of similarity between two things being compared. For example, a similarity value may be a number that indicates the overall similarity between a patient's biomarker profile using specific phenotype-related biomarkers and reference value ranges for the biomarkers in one or more control samples or a reference profile (e.g., the similarity to a “prostate cancer” biomarker profile). The similarity value may be expressed as a similarity metric, such as a correlation coefficient, or may simply be expressed as the expression level difference, or the aggregate of the expression level differences, between levels of biomarkers in a patient sample and a control sample or reference expression profile.

The terms “quantity”, “amount”, and “level” are used interchangeably herein and may refer to an absolute quantification of a molecule or an analyte in a sample, or to a relative quantification of a molecule or analyte in a sample, i.e., relative to another value such as relative to a reference value as taught herein, or to a range of values for the biomarker. These values or ranges can be obtained from a single patient or from a group of patients.

Biological sample. The term “sample” with respect to an individual encompasses blood and other liquid samples of biological origin, solid tissue samples such as a biopsy specimen or tissue cultures or cells derived or isolated therefrom and the progeny thereof. The definition also includes samples that have been manipulated in any way after their procurement, such as by treatment with reagents; washed; or enrichment for certain cell populations. The definition also includes samples that have been enriched for particular types of molecules, e.g., nucleic acids (cell-free DNA), polypeptides, etc.

The term “cfDNA sample” with respect to an individual encompasses samples such as blood or plasma samples comprising cfDNA obtained from the individual. The cfDNA samples can be obtained by any suitable method such as by venipuncture. The definition also includes samples that have been manipulated in any way after their procurement, such as by treatment with reagents, washed, centrifuged, or enriched for particular types of molecules (e.g., methylated cfDNA biomarkers).

Obtaining and assaying a sample. The term “assaying” is used herein to include the physical steps of manipulating a biological sample to generate data related to the sample. As will be readily understood by one of ordinary skill in the art, a biological sample must be “obtained” prior to assaying the sample. Thus, the term “assaying” implies that the sample has been obtained. The terms “obtained” or “obtaining” as used herein encompass the act of receiving an extracted or isolated biological sample. For example, a testing facility can “obtain” a biological sample in the mail (or via delivery, etc.) prior to assaying the sample. In some such cases, the biological sample was “extracted” or “isolated” from an individual by another party prior to mailing (i.e., delivery, transfer, etc.), and then “obtained” by the testing facility upon arrival of the sample. Thus, a testing facility can obtain the sample and then assay the sample, thereby producing data related to the sample.

The terms “obtained” or “obtaining” as used herein can also include the physical extraction or isolation of a biological sample from a subject. Accordingly, a biological sample can be isolated from a subject (and thus “obtained”) by the same person or same entity that subsequently assays the sample. When a biological sample is “extracted” or “isolated” from a first party or entity and then transferred (e.g., delivered, mailed, etc.) to a second party, the sample was “obtained” by the first party (and also “isolated” by the first party), and then subsequently “obtained” (but not “isolated”) by the second party. Accordingly, in some embodiments, the step of obtaining does not comprise the step of isolating a biological sample.

In some embodiments, the step of obtaining comprises the step of isolating a biological sample (e.g., a pre-treatment biological sample, a post-treatment biological sample, etc.). Methods and protocols for isolating various biological samples (e.g., a blood sample, a serum sample, a plasma sample, a biopsy sample, an aspirate, etc.) will be known to one of ordinary skill in the art and any convenient method may be used to isolate a biological sample.

It will be understood by one of ordinary skill in the art that in some cases, it is convenient to wait until multiple samples (e.g., a pre-treatment biological sample and a post-treatment biological sample) have been obtained prior to assaying the samples. Accordingly, in some cases an isolated biological sample (e.g., a pre-treatment biological sample, a post-treatment biological sample, etc.) is stored until all appropriate samples have been obtained. One of ordinary skill in the art will understand how to appropriately store a variety of different types of biological samples and any convenient method of storage may be used (e.g., refrigeration) that is appropriate for the particular biological sample. In some embodiments, a pre-treatment biological sample is assayed prior to obtaining a post-treatment biological sample. In some cases, a pre-treatment biological sample and a post-treatment biological sample are assayed in parallel. In some cases, multiple different post-treatment biological samples and/or a pre-treatment biological sample are assayed in parallel. In some cases, biological samples are processed immediately or as soon as possible after they are obtained.

The terms “determining”, “measuring”, “evaluating”, “assessing,” “assaying,” and “analyzing” are used interchangeably herein to refer to any form of measurement, and include determining if an element is present or not. These terms include both quantitative and/or qualitative determinations. Assaying may be relative or absolute. For example, “assaying” can be determining whether the methylation level or frequency is less than or “greater than or equal to” a particular threshold, (the threshold can be pre-determined or can be determined by assaying a control sample). On the other hand, “assaying to determine the methylation level” can mean determining a quantitative value (using any convenient metric) that represents the level of methylation at a CpG site. The level of methylation can be expressed in arbitrary units associated with a particular assay (e.g., fluorescence units, e.g., mean fluorescence intensity (MFI)), or can be expressed as an absolute value with defined units (e.g., number of methylated CpG sites in a cfDNA gene, frequency of methylation at a CpG site in cfDNA, etc.). Additionally, the level of methylation at a CpG site can be compared to the methylation level of one or more additional CpG sites to derive a normalized value that represents a normalized methylation level. The specific metric (or units) chosen is not crucial as long as the same units are used (or conversion to the same units is performed) when evaluating multiple samples from the same individual (e.g., samples taken at different points in time from the same individual). This is because the units cancel when calculating a fold-change (i.e., determining a ratio) in the methylation level from one sample to the next (e.g., samples taken at different points in time from the same individual).

As used herein, “methylation” refers to cytosine methylation at positions C5 or N4 of cytosine, the N6 position of adenine, or other types of nucleic acid methylation. In vitro amplified DNA is usually unmethylated because typical in vitro DNA amplification methods do not retain the methylation pattern of the amplification template. However, “unmethylated DNA” or “methylated DNA” can also refer to amplified DNA whose original template was unmethylated or methylated, respectively.

Accordingly, as used herein a “methylated nucleotide” or a “methylated nucleotide base” refers to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide. In another example, thymine contains a methyl moiety at position 5 of its pyrimidine ring; however, for purposes herein, thymine is not considered a methylated nucleotide when present in DNA since thymine is a typical nucleotide base of DNA.

As used herein, a “methylated nucleic acid molecule” refers to a nucleic acid molecule that contains one or more methylated nucleotides.

As used herein, a “methylation state”, “methylation profile”, and “methylation status” of a nucleic acid molecule refers to the presence of absence of one or more methylated nucleotide bases in the nucleic acid molecule. For example, a nucleic acid molecule containing a methylated cytosine is considered methylated (e.g., the methylation state of the nucleic acid molecule is methylated). A nucleic acid molecule that does not contain any methylated nucleotides is considered unmethylated.

The methylation state of a particular nucleic acid sequence (e.g., a gene marker or DNA region as described herein) can indicate the methylation state of every base in the sequence or can indicate the methylation state of a subset of the bases (e.g., of one or more cytosines) within the sequence, or can indicate information regarding regional methylation density within the sequence with or without providing precise information of the locations within the sequence the methylation occurs.

The methylation state of a nucleotide locus in a nucleic acid molecule refers to the presence or absence of a methylated nucleotide at a particular locus in the nucleic acid molecule. For example, the methylation state of a cytosine at the 7^{th} nucleotide in a nucleic acid molecule is methylated when the nucleotide present at the 7^{th} nucleotide in the nucleic acid molecule is 5-methylcytosine. Similarly, the methylation state of a cytosine at the 7^{th} nucleotide in a nucleic acid molecule is unmethylated when the nucleotide present at the 7^{th} nucleotide in the nucleic acid molecule is cytosine (and not 5-methylcytosine).

The methylation status can optionally be represented or indicated by a “methylation value” (e.g., representing a methylation frequency, fraction, ratio, percent, etc.). A methylation value can be generated, for example, by quantifying the amount of intact nucleic acid present following restriction digestion with a methylation dependent restriction enzyme or by comparing amplification profiles after bisulfite reaction or by comparing sequences of bisulfite-treated and untreated nucleic acids. Accordingly, a value, e.g., a methylation value, represents the methylation status and can thus be used as a quantitative indicator of methylation status across multiple copies of a locus. This is of particular use when it is desirable to compare the methylation status of a sequence in a sample to a threshold or reference value.

As used herein, “methylation frequency” or “methylation percent (%)” refer to the number of instances in which a molecule or locus is methylated relative to the number of instances the molecule or locus is unmethylated.

As such, the methylation state describes the state of methylation of a nucleic acid (e.g., a genomic sequence). In addition, the methylation state refers to the characteristics of a nucleic acid segment at a particular genomic locus relevant to methylation. Such characteristics include, but are not limited to, whether any of the cytosine (C) residues within this DNA sequence are methylated, the location of methylated C residue(s), the frequency or percentage of methylated C throughout any particular region of a nucleic acid, and allelic differences in methylation due to, e.g., difference in the origin of the alleles. The terms “methylation state”, “methylation profile”, and “methylation status” also refer to the relative concentration, absolute concentration, or pattern of methylated C or unmethylated C throughout any particular region of a nucleic acid in a biological sample. For example, if the cytosine (C) residue(s) within a nucleic acid sequence are methylated it may be referred to as “hypermethylated” or having “increased methylation”, whereas if the cytosine (C) residue(s) within a DNA sequence are not methylated it may be referred to as “hypomethylated” or having “decreased methylation”. Likewise, if the cytosine (C) residue(s) within a nucleic acid sequence are methylated as compared to another nucleic acid sequence (e.g., from a different region or from a different individual, etc.) that sequence is considered hypermethylated or having increased methylation compared to the other nucleic acid sequence. Alternatively, if the cytosine (C) residue(s) within a DNA sequence are not methylated as compared to another nucleic acid sequence (e.g., from a different region or from a different individual, etc.) that sequence is considered hypomethylated or having decreased methylation compared to the other nucleic acid sequence. Additionally, the term “methylation pattern” as used herein refers to the collective sites of methylated and unmethylated nucleotides over a region of a nucleic acid. Two nucleic acids may have the same or similar methylation frequency or methylation percent but have different methylation patterns when the number of methylated and unmethylated nucleotides are the same or similar throughout the region but the locations of methylated and unmethylated nucleotides are different. Sequences are said to be “differentially methylated” or as having a “difference in methylation” or having a “different methylation state” when they differ in the extent (e.g., one has increased or decreased methylation relative to the other), frequency, or pattern of methylation. The term “differential methylation” refers to a difference in the level or pattern of nucleic acid methylation in a cancer positive sample as compared with the level or pattern of nucleic acid methylation in a cancer negative sample. It may also refer to the difference in levels or patterns between patients that have recurrence of cancer after surgery versus patients who not have recurrence. Differential methylation and specific levels or patterns of DNA methylation are prognostic and predictive biomarkers, e.g., once the correct cut-off or predictive characteristics have been defined.

The methylation state frequency can be used to describe a population of individuals or a sample from a single individual. For example, a nucleotide locus having a methylation state frequency of 50% is methylated in 50% of instances and unmethylated in 50% of instances. Such a frequency can be used, for example, to describe the degree to which a nucleotide locus or nucleic acid region is methylated in a population of individuals or a collection of nucleic acids. Thus, when methylation in a first population or pool of nucleic acid molecules is different from methylation in a second population or pool of nucleic acid molecules, the methylation state frequency of the first population or pool will be different from the methylation state frequency of the second population or pool. Such a frequency also can be used, for example, to describe the degree to which a nucleotide locus or nucleic acid region is methylated in a single individual. For example, such a frequency can be used to describe the degree to which a group of cells from a tissue sample are methylated or unmethylated at a nucleotide locus or nucleic acid region.

As used herein a “nucleotide locus” refers to the location of a nucleotide in a nucleic acid molecule. A nucleotide locus of a methylated nucleotide refers to the location of a methylated nucleotide in a nucleic acid molecule.

Typically, methylation of human DNA occurs on a dinucleotide sequence including an adjacent guanine and cytosine where the cytosine is located 5′ of the guanine (also termed CpG dinucleotide sequences). Most cytosines within the CpG dinucleotides are methylated in the human genome, however some remain unmethylated in specific CpG dinucleotide rich genomic regions, known as CpG islands (see, e.g., Antequera et al. (1990) Cell 62: 503-514).

As used herein, a “CpG island” refers to a G:C-rich region of genomic DNA containing an increased number of CpG dinucleotides relative to total genomic DNA. A CpG island can be at least 100, 200, or more base pairs in length, where the G:C content of the region is at least 50% and the ratio of observed CpG frequency over expected frequency is 0.6; in some instances, a CpG island can be at least 500 base pairs in length, where the G:C content of the region is at least 55%) and the ratio of observed CpG frequency over expected frequency is 0.65. The observed CpG frequency over expected frequency can be calculated according to the method provided in Gardiner-Garden et al (1987) J. Mol. Biol. 196: 261-281. For example, the observed CpG frequency over expected frequency can be calculated according to the formula R=(A×B)/(C×D), where R is the ratio of observed CpG frequency over expected frequency, A is the number of CpG dinucleotides in an analyzed sequence, B is the total number of nucleotides in the analyzed sequence, C is the total number of C nucleotides in the analyzed sequence, and D is the total number of G nucleotides in the analyzed sequence. Methylation state is typically determined in CpG islands, e.g., at promoter regions. It will be appreciated though that other sequences in the human genome are prone to DNA methylation such as CpA and CpT (see, e.g., Ramsahoye (2000) Proc. Natl. Acad. Sci. USA 97: 5237-5242; Salmon and Kaye (1970) Biochim. Biophys. Acta. 204: 340-351; Grafstrom (1985) Nucleic Acids Res. 13: 2827-2842; Nyce (1986) Nucleic Acids Res. 14: 4353-4367; Woodcock (1987) Biochem. Biophys. Res. Commun. 145: 888-894).

As used herein, a reagent that modifies a nucleotide of the nucleic acid molecule as a function of the methylation state of the nucleic acid molecule, or a methylation-specific reagent, refers to a compound or composition or other agent that can change the nucleotide sequence of a nucleic acid molecule in a manner that reflects the methylation state of the nucleic acid molecule. Methods of treating a nucleic acid molecule with such a reagent can include contacting the nucleic acid molecule with the reagent, coupled with additional steps, if desired, to accomplish the desired change of nucleotide sequence. Such a change in the nucleic acid molecule's nucleotide sequence can result in a nucleic acid molecule in which each methylated nucleotide is modified to a different nucleotide. Such a change in the nucleic acid nucleotide sequence can result in a nucleic acid molecule in which each unmethylated nucleotide is modified to a different nucleotide. Such a change in the nucleic acid nucleotide sequence can result in a nucleic acid molecule in which each of a selected nucleotide which is unmethylated (e.g., each unmethylated cytosine) is modified to a different nucleotide. Use of such a reagent to change the nucleic acid nucleotide sequence can result in a nucleic acid molecule in which each nucleotide that is a methylated nucleotide (e.g., each methylated cytosine) is modified to a different nucleotide. As used herein, use of a reagent that modifies a selected nucleotide refers to a reagent that modifies one nucleotide of the four typically occurring nucleotides in a nucleic acid molecule (C, G, T, and A for DNA and C, G, U, and A for RNA), such that the reagent modifies the one nucleotide without modifying the other three nucleotides. In one exemplary embodiment, such a reagent modifies an unmethylated selected nucleotide to produce a different nucleotide. In another exemplary embodiment, such a reagent can deaminate unmethylated cytosine nucleotides. An exemplary reagent is bisulfite.

As used herein, the term “bisulfite reagent” refers to a reagent comprising in some embodiments bisulfite, disulfite, hydrogen sulfite, or combinations thereof to distinguish between methylated and unmethylated cytidines, e.g., in CpG dinucleotide sequences.

The term “methylation assay” refers to any assay or method for determining the methylation state of one or more CpG dinucleotide sequences within a sequence of a nucleic acid. Exemplary methylation assays include, without limitation, methylation-sensitive arbitrarily-primed polymerase chain reaction (MS AP-PCR), methylation-sensitive single nucleotide primer extension (Ms-SNuPE), methylation-specific PCR (MSP), methylation-sensitive DNA restriction enzyme analysis, restriction enzyme-based sequencing, restriction enzyme-based microarray analysis, combined bisulfite restriction analysis (COBRA), methylated CpG island amplification (MCA), methylated CpG island amplification and microarray (MCAM), HpaII tiny fragment enrichment by ligation-mediated PCR (HELP), bisulfite sequencing, bisulfite microarray analysis, methylation-specific pyrosequencing, HELP-sequencing (HELP-seq), TET-assisted pyridine borane sequencing (TAPS), GlaI hydrolysis and ligation adapter dependent PCR (GLAD-PCR), methylated DNA immunoprecipitation-sequencing (MeDIP-Seq), or methylated DNA immunoprecipitation-microarray analysis (MeDI P-chip), Southern blotting with methyl-sensitive restriction enzymes, and methylation-specific giant magnetoresistive sensor-based microarray analysis.

Bisulfite sequencing uses bisulfite treatment of DNA before sequencing to detect methylation sites. Treatment of DNA with bisulfite converts cytosine residues to uracil, but does not affect methylated cytosine residues. After bisulfite treatment, the only cytosines remaining in the DNA are methylated cytosines. Thus, sequencing the DNA after bisulfite treatment reveals the methylation status of individual cytosine residues at single-nucleotide resolution.

The MS AP-PCR assay uses methylation-sensitive restriction enzymes to digest DNA and PCR with CG-rich primers to selectively amplify regions that contain CpG dinucleotides (see, e.g., Gonzalgo et al. (1997) Cancer Research 57: 594-599, herein incorporated by reference).

The MethyLight assay uses bisulfite-dependent, quantitative fluorescence-based real-time PCR with methylation-specific priming and methylation-specific fluorescent probing for detection of DNA methylation. Digital MethyLight combines the MethyLight assay with digital PCR to allow detection of individual methylated molecules (see, e.g., Eads et al. (1999) Cancer Res. 59: 2302-2306, Campan et al. (2018) Methods Mol. Biol. 1708:497-513).

The HeavyMethy assay uses methylation specific blocking probes (also referred to herein as blockers) covering CpG positions between, or covered by, amplification primers to enable methylation-specific selective amplification of a nucleic acid sample.

The HeavyMethyl MethyLight assay is a variation of the MethyLight™ assay, wherein the MethyLight™ assay is combined with methylation specific blocking probes covering CpG positions between the amplification primers.

The Ms-SNuPE assay uses bisulfite treatment of DNA combined with PCR using primers designed to hybridize immediately upstream of the CpG site(s) and electrophoresis of amplicons on polyacrylamide gels for visualization and quantitation. Treatment of DNA (genomic or cfDNA) with sodium bisulfite causes unmethylated cytosines to be converted to uracils, During the PCR step, uracil is replicated as thymine, and methylcytosine is replicated as cytosine during amplification. The ratio of methylated versus unmethylated cytosine (C versus T) at the original CpG sites can be determined by incubating the gel-isolated PCR product, primer(s), and Taq polymerase with either [³²P]dCTP or [³²P]TTP followed by denaturing polyacrylamide gel electrophoresis and phosphorimage analysis. Ms-SNuPE primers can also be designed to incorporate either [³²P]dATP or [³²P]dGTP into the opposite strand to assess methylation status depending on which CpG site is analyzed (see, e.g., Gonzalgo & Jones (1997) Nucleic Acids Res. 25: 2529-2531; Gonzalgo et al. (2007) Nat. Protoc. 2(8):1931-6).

The MSP assay uses bisulfite treatment of DNA for conversion of non-methylated cytosines to uracils and subsequent amplification with primers specific for methylated versus unmethylated DNA (see, e.g., Herman et al. (1996) Proc. Natl. Acad. Sci. USA 93: 9821-9826, and U.S. Pat. No. 5,786,146).

The COBRA assay uses bisulfite treatment of DNA for conversion of non-methylated cytosines to uracils, locus-specific PCR amplification of the bisulfite-converted DNA, restriction digestion, electrophoresis an analysis of restriction patterns on a gel (see, e.g., Xiong & Laird (1997) Nucleic Acids Res. 25: 2532-2534; Bilichak et al. (2017) Methods Mol. Biol. 1456:63-71).

The MCA assay uses methylation-sensitive restriction enzymes to digest DNA, followed by adaptor ligation and PCR to selectively amplify methylated CpG-rich sequences (see, e.g., Toyota et al. (1999) Cancer Res. 59: 2307-12, and WO 00/26401A1).

The MCAM assay uses MCA in combination with a CpG island microarray to detect DNA methylation in a high-throughput fashion (see, e.g., Estecio et al. (2007) Genome Res. 17(10):1529-1536).

The HELP assay uses the methylation-sensitive restriction enzyme, HpaII, to cut DNA, and a methylation-insensitive isoschizomer, MspI, as a control. Microarray analysis is performed with microarrays containing probes designed to detect the HpaII/MspI fragments. HELP-seq combines the HELP assay with massively parallel sequencing of DNA methylation sites (see, e.g., Greally (2018) Methods Mol. Biol. 1708:191-207; Suzuki et al. (2010) Methods 52(3):218-22).

The GLAD-PCR assay uses a site-specific methyl-directed DNA-endonucleases that cleave only methylated DNA, followed by ligation of DNA fragments to universal adapters for high-throughput PCR (see, e.g., Malyshev et al. (2020) Acta Naturae 12(3):124-133; Russian Patent RU 2525710).

The MeDIP assay uses an antibody against 5-methylcytosine to immunoprecipitate methylated DNA fragments. This technique can be combined with high-throughput DNA detection methods using microarray hybridization (MeDIP-chip) or next-generation sequencing (MeDIP-seq). See, e.g., Weber et al. (2005) Nat. Genet. 37 (8): 853-862, Palmke et al. (2011) Methods 53(2):175-184, Quackenbush et al. (2008) Cancer Res. 68(6):1786-1796, Zhu et al. (2019) Analyst 144(6):1904-1915, Yang et al. (2014) Life Sci. 113(1-2):45-54.

TET-assisted pyridine borane sequencing (TAPS) uses the ten-eleven translocation (TET) enzyme to catalyze oxidation of 5-methylcytosine and 5-hydroxymethylcytosine to 5-carboxylcytosine, followed by pyridine borane reduction to produce dihydrouracil. Unmodified cytosine is not affected. See, e.g., Liu et al. (2019) Nat Biotechnol. 37:424-429.

Methylation-specific giant magnetoresistive sensor-based microarray analysis combines methylation specific PCR and melt curve analysis on a giant magnetoresistive (GMR) biosensor. The GMR biosensor comprises synthetic DNA probes that target methylated or unmethylated CpG sites in the PCR amplicons. After hybridization of the PCR amplicons to the GMR biosensor, the difference in melting temperature (Tm) between the two types of probes is measured. See, e.g., Rizzi et al. (2017) ACS Nano. 11(9): 8864-8870, Nesvet et al. (2019) Biosens Bioelectron 124-125:136-142.

Southern Blotting can also be used to detect DNA methylation. The DNA is first digested with methylation-sensitive restriction enzymes, and the restriction fragments are analyzed by Southern Blot.

As used herein, a “selected nucleotide” refers to one nucleotide of the four typically occurring nucleotides in a nucleic acid molecule (C, G, T, and A for DNA and C, G, U, and A for RNA), and can include methylated derivatives of the typically occurring nucleotides (e.g., when C is the selected nucleotide, both methylated and unmethylated C are included within the meaning of a selected nucleotide), whereas a methylated selected nucleotide refers specifically to a methylated typically occurring nucleotide and an unmethylated selected nucleotides refers specifically to an unmethylated typically occurring nucleotide.

The terms “methylation-specific restriction enzyme” or “methylation-sensitive restriction enzyme” refers to an enzyme that selectively digests a nucleic acid dependent on the methylation state of its recognition site. In the case of a restriction enzyme that specifically cuts if the recognition site is not methylated or is hemimethylated, the cut will not take place or will take place with a significantly reduced efficiency if the recognition site is methylated. In the case of a restriction enzyme that specifically cuts if the recognition site is methylated, the cut will not take place or will take place with a significantly reduced efficiency if the recognition site is not methylated. Preferred are methylation-specific restriction enzymes, the recognition sequence of which contains a CG dinucleotide (for instance a recognition sequence such as CGCG or CCCGGG). Further preferred for some embodiments are restriction enzymes that do not cut if the cytosine in this dinucleotide is methylated at the carbon atom C5.

As used herein, a “different nucleotide” refers to a nucleotide that is chemically different from a selected nucleotide, typically such that the different nucleotide has Watson-Crick base-pairing properties that differ from the selected nucleotide, whereby the typically occurring nucleotide that is complementary to the selected nucleotide is not the same as the typically occurring nucleotide that is complementary to the different nucleotide. For example, when C is the selected nucleotide, U or T can be the different nucleotide, which is exemplified by the complementarity of C to G and the complementarity of U or T to A. As used herein, a nucleotide that is complementary to the selected nucleotide or that is complementary to the different nucleotide refers to a nucleotide that base-pairs, under high stringency conditions, with the selected nucleotide or different nucleotide with higher affinity than the complementary nucleotide's base-paring with three of the four typically occurring nucleotides. An example of complementarity is Watson-Crick base pairing in DNA (e.g., A-T and C-G) and RNA (e.g., A-U and C-G). Thus, for example, G base-pairs, under high stringency conditions, with higher affinity to C than G base-pairs to G, A, or T and, therefore, when C is the selected nucleotide, G is a nucleotide complementary to the selected nucleotide.

As used herein, the “sensitivity” of a given marker refers to the percentage of samples that report a DNA methylation value above a threshold value that distinguishes between neoplastic and non-neoplastic samples. In some embodiments, a positive is defined as a histology-confirmed neoplasia that reports a DNA methylation value above a threshold value (e.g., the range associated with disease), and a false negative is defined as a histology-confirmed neoplasia that reports a DNA methylation value below the threshold value (e.g., the range associated with no disease). The value of sensitivity, therefore, reflects the probability that a DNA methylation measurement for a given marker obtained from a known diseased sample will be in the range of disease-associated measurements. As defined here, the clinical relevance of the calculated sensitivity value represents an estimation of the probability that a given marker would detect the presence of a clinical condition when applied to a subject with that condition.

As used herein, the “specificity” of a given marker refers to the percentage of non-neoplastic samples that report a DNA methylation value below a threshold value that distinguishes between neoplastic and non-neoplastic samples. In some embodiments, a negative is defined as a histology-confirmed non-neoplastic sample that reports a DNA methylation value below the threshold value (e.g., the range associated with no disease) and a false positive is defined as a histology-confirmed non-neoplastic sample that reports a DNA methylation value above the threshold value (e.g., the range associated with disease). The value of specificity, therefore, reflects the probability that a DNA methylation measurement for a given marker obtained from a known non-neoplastic sample will be in the range of non-disease associated measurements. As defined here, the clinical relevance of the calculated specificity value represents an estimation of the probability that a given marker would detect the absence of a clinical condition when applied to a patient without that condition.

“Diagnosis” as used herein generally includes determination as to whether a subject is likely affected by a given disease, disorder or dysfunction. The skilled artisan often makes a diagnosis on the basis of one or more diagnostic indicators, i.e., a biomarker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

“Providing an analysis” is used herein to refer to the delivery of an oral or written analysis (i.e., a document, a report, etc.). A written analysis can be a printed or electronic document. A suitable analysis (e.g., an oral or written report) provides any or all of the following information: identifying information of the subject (name, age, etc.), a description of what type of sample(s) was used and/or how it was used, the technique used to assay the sample, the results of the assay (e.g., the level or frequency of cfDNA CpG methylation as measured and/or the fold-change of a level or frequency of cfDNA CpG methylation over time or in a post-treatment assay compared to a pre-treatment assay), the assessment as to whether the individual is determined to have prostate cancer or a recurrence of prostate cancer, a recommendation for treatment (e.g., a particular anti-cancer therapy), and/or to continue or alter therapy, a recommended strategy for additional therapy, an assessment as to whether the individual is determined to be responsive or not responsive to an anti-cancer treatment (e.g., abiraterone acetate), etc. The report can be in any format including, but not limited to printed information on a suitable medium or substrate (e.g., paper); or electronic format. If in electronic format, the report can be in any computer readable medium, e.g., diskette, compact disk (CD), flash drive, and the like, on which the information has been recorded. In addition, the report may be present as a website address which may be used via the internet to access the information at a remote site.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

It will be apparent to one of ordinary skill in the art that various changes and modifications can be made without departing from the spirit or scope of the invention.

Methylated cfDNA Biomarkers and Diagnostic Methods

Hypermethylation of CpG-islands in regulatory regions of promoters and/or the first exons in a variety of genes is associated with a variety of cancers. Layered analysis of methylated biomarkers (LAMB) was used to identify methylated cell-free DNA (cfDNA) biomarkers associated with prostate cancer (see Examples). The identified prostate cancer biomarkers include cfDNA methylated at one or more CpG sites in promoter regions of the genes, APC, CD44, PYCARD, RARB, and RBP1. Increased frequency or levels of methylation at CpG sites in these biomarker genes are commonly found in prostate cancer tumors. In particular, increased frequency or levels of methylation at one or more CpG sites selected from cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124 (locations of these CpG sites are given in the Illumina HumanMethylation450K Manifest), and CpG sites located within 200 nucleotides thereof is associated with prostate cancer. Accordingly, monitoring the frequency or levels of methylation of these CpG sites is useful for prognosis, diagnosis, therapy selection, and monitoring treatment of prostate cancer.

In certain embodiments, a panel of methylated cfDNA biomarkers for use in diagnosis of prostate cancer is provided. Biomarker panels of any size can be used in the practice of the subject methods. Biomarker panels for diagnosing prostate cancer typically comprise at least 2 methylated cfDNA biomarkers and up to 20 methylated cfDNA biomarkers, including any number of biomarkers in between, such as 2, 3, 4, 5, 6, 7, 8, 9, 10, 11, 12, 13, 14, 15, 16, 17, 18, 19, or 20 methylated cfDNA biomarkers. In certain embodiments, the biomarker panel comprises at least 2, or at least 3, or at least 4, or at least 5, or at least 6, or at least 7, or at least 8, or at least 9, or at least 10, or at least 11, or at least 12, or at least 13, or at least 14, or at least 5, or at least 16, or at least 17, or at least 18, or at least 19, or at least 20 or more methylated cfDNA biomarkers. In some embodiments, the biomarker panel comprises or consists of cfDNA biomarkers with methylation at one or more CpG sites selected from cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124, and CpG sites located within 200 nucleotides thereof. In some embodiments, the biomarker panel comprises or consists of cfDNA biomarkers with methylation at the CpG sites: cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124. Although smaller biomarker panels are usually more economical, larger biomarker panels (i.e., greater than 20 biomarkers) have the advantage of providing more detailed information and can also be used in the practice of the subject methods.

A sample comprising methylated cfDNA (i.e., a “cfDNA sample”) is obtained from the subject. The sample is typically a blood or plasma sample comprising cfDNA taken from the subject. A “control” sample, as used herein, refers to a cfDNA sample from a subject that is not diseased. That is, a control sample is obtained from a normal or healthy subject (e.g., an individual known to not have prostate cancer). A cfDNA sample can be obtained from a subject by conventional techniques. For example, blood samples can be obtained by venipuncture according to methods well known in the art.

When analyzing the frequency or levels of methylation at CpG sites in a cfDNA sample from a subject, the reference value ranges used for comparison can represent the frequency or levels of DNA methylation at CpG sites in a cfDNA sample from one or more subjects without prostate cancer (i.e., normal or healthy control). Alternatively, the reference values can represent the frequency or levels of methylation at CpG sites in cfDNA samples from one or more subjects with prostate cancer, wherein similarity to the reference value ranges indicates the subject has prostate cancer.

In some cases, combinations of methylated cfDNA biomarkers are used in the subject methods. In some such cases, the levels of all measured biomarkers must change (as described above) in order for the diagnosis to be made. In some embodiments, only some biomarkers are used in the methods described herein. For example, a single biomarker, 2 biomarkers, 3 biomarkers, 4 biomarkers, 5 biomarkers, 6 biomarkers, 7 biomarkers, 8 biomarkers, 9 biomarkers, 10 biomarkers, 11 biomarkers, 12 biomarkers, 13 biomarkers, 14 biomarkers, 15 biomarkers, 16 biomarkers, 17 biomarkers, 18 biomarkers, 19 biomarkers, or 20 biomarkers can be used in any combination. In other embodiments, all the biomarkers are used. The quantitative values may be combined in linear or non-linear fashion to calculate one or more risk scores for prostate cancer for the individual. In some embodiments, a geometric mean score is calculated from the gene methylation frequency profile for 2, 3, 4, or all 5 of the APC, CD44, PYCARD, RARB, and RBP1 genes, wherein the geometric mean score indicates whether or not the individual has prostate cancer. The geometric mean score may further distinguish between a subject who has prostate cancer versus a subject who does not have prostate cancer.

The methods described herein may be used to determine an appropriate treatment regimen for a patient and, in particular, whether a patient should be treated for prostate cancer. For example, a patient is selected for treatment for prostate cancer if the patient has a positive diagnosis for prostate cancer based on a cfDNA methylation profile, as described herein. In some cases, the diagnostic methods described herein may be used by themselves or combined with medical imaging to confirm the diagnosis and further evaluate the extent of cancerous disease (how far and where the cancer has spread) to aid in determining prognosis and evaluating optimal strategies for treatment (e.g., surgery, androgen deprivation therapy, radionuclide therapy, chemotherapy, targeted therapy, immunotherapy, biologic therapy, etc.). Exemplary medical imaging techniques include, without limitation, magnetic resonance imaging (MRI), positron emission tomography (PET), single photon emission computed tomography (SPECT), computed tomography (CT), ultrasound imaging (UI), optical imaging (OI), photoacoustic imaging (PI), fluoroscopy, and fluorescence imaging.

In some embodiments, the methylated cfDNA biomarkers are used in combination with other biomarkers for diagnosing prostate cancer, such as prostate-specific antigen (PSA), kallikrein 2, and prostate cancer antigen 3 (PCA3). For example, blood levels of PSA and/or kallikrein 2 or urine levels of PCA3 can be monitored in addition to methylation of the cfDNA biomarkers. Elevated levels of PSA and/or kallikrein 2 in blood and/or elevated levels of PCA3 in urine in combination with increased frequency of methylation at the one or more CpG sites in the one or more genes selected from the group consisting of APC, CD44, PYCARD, RARB, and RBP1 in a cfDNA sample from a patient compared to reference value ranges for frequency of methylation at the one or more CpG sites in a control cfDNA sample indicate that a patient has a positive diagnosis for the prostate cancer.

Exemplary treatments for prostate cancer include, without limitation, tumor surgical resection, prostatectomy, external beam radiation therapy, particle therapy, brachytherapy, cryosurgery, high-intensity focused ultrasound, hormonal therapy, chemotherapy, targeted therapy, and immunotherapy. Medications used to treat prostate cancer include, without limitation, antiandrogens including, without limitation, flutamide, nilutamide, bicalutamide, enzalutamide, apalutamide, and cyproterone acetate; medications that block the production of adrenal androgens including, without limitation, ketoconazole and aminoglutethimide; GnRH antagonists including, without limitation, abarelix and degarelixas; GnRH agonists including, without limitation, leuprorelin and goserelin; CYP17 inhibitors including, without limitation, abiraterone acetate; estrogens including, without limitation, diethylstilbestrol, fosfestrol, ethinylestradiol, ethinylestradiol sulfonate, polyestradiol phosphate, and estradiol undecylate; 5-alpha-reductase inhibitors, including, without limitation, finasteride and dutasteride; chemotherapeutic agents, including, without limitation, docetaxel, cabazitaxel, and immunotherapeutic agents, including, without limitation, Sipuleucel-T and bevacizumab; or a combination thereof.

For patients without systemic disease, only localized adjuvant therapy (e.g., radiation therapy of the prostate bed) or a short course of anti-androgen therapy may be administered. For patients with no evidence of disease, adjuvant therapy is generally not recommended in order to avoid treatment-related side effects such as metabolic syndrome (e.g., hypertension, diabetes and/or weight gain), osteoporosis, proctitis, incontinence or impotence. Patients with no evidence of disease could be designated for watchful waiting, monitoring for disease progression, or no treatment. Patients without systemic disease, but who have successive PSA increases, could be designated for watchful waiting, increased monitoring, or lower dose or shorter duration anti-androgen therapy. Patients with systemic disease after a prostatectomy, may undergo additional treatment such as with adjuvant chemotherapy (e.g., docetaxel, mitoxantrone and prednisone), systemic radiation therapy (e.g., samarium or strontium) and/or anti-androgen therapy (e.g., surgical castration, finasteride, dutasteride, abiraterone). Metastatic castration-resistant prostate cancer may be treated, for example, with abiraterone acetate (AA). In some cases, AA is administered in combination with a corticosteroid such as prednisone or a chemotherapeutic agent such as docetaxel. Patients may be treated with anti-androgen therapy alone or in combination with radiation therapy in order to eliminate micro-metastatic disease, which cannot be readily detected clinically. Such patients may be closely monitored for signs of disease progression.

The cfDNA biomarkers can be used for such monitoring of prostate cancer in a patient. For example, a first cfDNA sample can be obtained from the patient at a first time point and a second cfDNA sample can be obtained from the patient at a second time point. In some embodiments, the patient is monitored for prostate cancer by detecting methylation at one or more CpG sites in one or more genes of the cfDNA in the first cfDNA sample and the second cfDNA sample, wherein the one or more genes are selected from the group consisting of APC, CD44, PYCARD, RARB, and RBP1, wherein detection of increased frequency of methylation of the CpG sites in the one or more genes selected from the group consisting of APC, CD44, PYCARD, RARB, and RBP1 in the second cfDNA sample compared to the first cfDNA sample indicate that the prostate cancer is progressing, and detection of decreased frequency of methylation of the CpG sites in the one or more genes selected from the group consisting of APC, CD44, PYCARD, RARB, and RBP1 in the second cfDNA sample compared to the first cfDNA sample indicate that the prostate cancer is not progressing. In some embodiments, the patient is monitored over a period of time by repeatedly collecting cfDNA samples at intervals and analyzing the cfDNA to determine whether or not the prostate cancer is progressing. Prostate cancer at any stage of progression can be monitored, including primary tumors, metastases, or recurrences.

The subject methods are especially useful for diagnosing or monitoring a patient, as described herein, if the patient has risk factors that make the patient susceptible to developing prostate cancer, such as obesity, age (e.g., low risk for men under 45, moderate risk for men in their 60s, high risk for men over 70), family history (e.g., family member or close relative with prostate cancer), a genetic susceptibility, an underlying condition or disease, or an environmental exposure to a carcinogen. For example, mutations in the BRCA1 and BRCA2 genes, the hereditary prostate cancer gene 1 (HPC1), the androgen receptor, the vitamin D receptor, and at the single nucleotide polymorphisms (SNPs), rs10993994, rs78943174, and rs35148638, and TMPRSS2-ETS gene family fusions (e.g., TMPRSS2-ERG or TMPRSS2-ETV1/4) are associated with increased risk of prostate cancer. Exemplary conditions and diseases that increase susceptibility to prostate cancer include, but are not limited to, prostatitis (e.g., from infection or inflammation), sexually transmitted infections such as chlamydia, gonorrhea, or syphilis, and papilloma virus infection. Exposure of veterans to Agent Orange has also been linked to increased risk of developing prostate cancer.

The subject methods may also be used for assaying pre-treatment and post-treatment cfDNA samples obtained from an individual to determine whether the individual is responsive or not responsive to a treatment. For example, a first cfDNA sample can be obtained from a subject before the subject undergoes a therapy, and a second cfDNA sample can be obtained from the subject after the subject undergoes the therapy. In one embodiment, the efficacy of a treatment of a patient for prostate cancer is monitored by measuring the frequency or levels of methylation at one or more CpG sites selected from cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124, and CpG sites located within 200 nucleotides thereof, in the first cfDNA sample and the second cfDNA sample; and evaluating the efficacy of the treatment, wherein detection of increased frequency or levels of methylation at one or more CpG sites selected from cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124, and CpG sites located within 200 nucleotides thereof, in the second cfDNA sample compared to the first cfDNA sample indicate that the patient is worsening or not responding to the treatment, and detection of decreased frequency or levels of methylation at one or more CpG sites selected from cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124, and CpG sites located within 200 nucleotides thereof, in the second cfDNA sample compared to the first cfDNA sample indicate that the patient is improving. In certain embodiments, the cfDNA biomarkers described herein are used to monitor treatment with abiraterone acetate, either alone, or in combination with a corticosteroid such as prednisone or a chemotherapeutic agent such as docetaxel.

The frequency or level of methylation of a cfDNA biomarker gene in a pre-treatment sample can be referred to as a “pre-treatment value” because the first sample is isolated from the individual prior to the administration of the therapy (i.e., “pre-treatment”). The frequency or level of methylation of a cfDNA biomarker gene in the pre-treatment sample can also be referred to as a “baseline value” because this value is the value to which “post-treatment” values are compared. In some cases, the baseline value (i.e., “pre-treatment value”) is determined by determining the frequency or level of methylation of a cfDNA biomarker gene in multiple (i.e., more than one, e.g., two or more, three or more, for or more, five or more, etc.) pre-treatment samples. In some cases, the multiple pre-treatment samples are isolated from an individual at different time points in order to assess natural fluctuations in biomarker levels prior to treatment. As such, in some cases, one or more (e.g., two or more, three or more, for or more, five or more, etc.) pre-treatment samples are isolated from the individual. In some embodiments, all of the pre-treatment samples will be the same type of sample (e.g., a blood sample). In some cases, two or more pre-treatment samples are pooled prior to determining the level of the biomarker in the samples. In some cases, the frequency or level of methylation of a cfDNA biomarker gene is determined separately for two or more pre-treatment samples and a “pre-treatment value” is calculated by averaging the separate measurements.

A post-treatment sample is isolated from an individual after the administration of a therapy. Thus, the frequency or level of methylation of a cfDNA biomarker gene in a post-treatment sample can be referred to as a “post-treatment value”. In some embodiments, the frequency or level of methylation of a cfDNA biomarker gene is measured in additional post-treatment samples (e.g., a second, third, fourth, fifth, etc. post-treatment sample). Because additional post-treatment samples are isolated from the individual after the administration of a treatment, the levels of a biomarker in the additional samples can also be referred to as “post-treatment values.”

The term “responsive” as used herein means that the treatment is having the desired effect such as an anti-tumor effect. For example, a positive therapeutic response would refer to one or more of the following improvements in the disease: (1) reduction in tumor size; (2) reduction in the number of cancer cells; (3) inhibition (i.e., slowing to some extent, preferably halting) of tumor growth; (4) inhibition (i.e., slowing to some extent, preferably halting) of cancer cell infiltration into peripheral organs; (5) inhibition (i.e., slowing to some extent, preferably halting) of tumor metastasis; and (6) some extent of relief from one or more symptoms associated with the cancer. When the individual does not improve in response to the treatment, it may be desirable to seek a different therapy or treatment regime for the individual.

The determination that an individual has prostate cancer is an active clinical application of the correlation between the frequency or level of methylation of one or more cfDNA biomarker genes and the disease. For example, “determining” requires the active step of reviewing the data, which is produced during the active assaying step(s), and resolving whether an individual does or does not have prostate cancer or is responding or not responding to a therapy for treatment of prostate cancer. Additionally, in some cases, a decision is made to proceed with the current treatment (i.e., therapy), or instead to alter the treatment. In some cases, the subject methods include the step of continuing therapy or altering therapy.

The term “continue treatment” (i.e., continue therapy) is used herein to mean that the current course of treatment (e.g., continued administration of a therapy) is to continue. If the current course of treatment is not effective in treating prostate cancer, the treatment may be altered. “Altering therapy” is used herein to mean “discontinuing therapy” or “changing the therapy” (e.g., changing the type of treatment, changing the particular dose and/or frequency of administration of medication, e.g., increasing the dose and/or frequency). In some cases, therapy can be altered until the individual is deemed to be responsive. In some embodiments, altering therapy means changing which type of treatment is administered, discontinuing a particular treatment altogether, etc.

As a non-limiting illustrative example, a patient may be initially treated with androgen deprivation therapy. Then to “continue treatment” would be to continue with this type of treatment. If the current course of treatment is not effective, the treatment may be altered, e.g., increasing dosage or frequency of a treatment for prostate cancer, changing to a different treatment, or starting palliative care for the patient. Switching treatment might involve, for example, administering a different type of androgen deprivation therapy (e.g., administering an antiandrogen after surgical castration) or administering a different type of anti-cancer therapy such as chemotherapy, radiation therapy, immunotherapy, etc.

In other words, the frequency or level of methylation of one or more cfDNA biomarker genes may be monitored in order to determine when to continue therapy and/or when to alter therapy. As such, a post-treatment cfDNA sample can be isolated after any of the administrations and the cfDNA sample can be assayed to determine the frequency or level of methylation of one or more cfDNA biomarker genes. Accordingly, the subject methods can be used to determine whether an individual being treated for prostate cancer is responsive or is maintaining responsiveness to a treatment.

The therapy can be administered to an individual any time after a pre-treatment cfDNA sample is isolated from the individual, but it is preferable for the therapy to be administered simultaneous with or as soon as possible (e.g., about 7 days or less, about 3 days or less, e.g., 2 days or less, 36 hours or less, 1 day or less, 20 hours or less, 18 hours or less, 12 hours or less, 9 hours or less, 6 hours or less, 3 hours or less, 2.5 hours or less, 2 hours or less, 1.5 hours or less, 1 hour or less, 45 minutes or less, 30 minutes or less, 20 minutes or less, 15 minutes or less, 10 minutes or less, 5 minutes or less, 2 minutes or less, or 1 minute or less) after a pre-treatment cfDNA sample is isolated (or, when multiple pre-treatment cfDNA samples are isolated, after the final pre-treatment cfDNA sample is isolated).

In some cases, more than one type of therapy may be administered to the individual. For example, a subject who has prostate cancer may undergo a prostatectomy or androgen deprivation therapy followed by administration of a chemotherapeutic agent, immunotherapeutic, or biologic agent. Systemic therapy may be administered if the cancer spreads beyond the prostate or undergoes metastasis.

The subject methods can also be used to determine if an individual with prostate cancer is likely to be responsive to treatment with abiraterone acetate (17-(3-pyridinyl)androsta-5,16-dien-3β-ol acetate). In some embodiments, the frequency of methylation is measured at one or more CpG sites in at least one biomarker gene selected from the group consisting of RARB and RBP1 in cell-free DNA (cfDNA) from a blood sample collected from the individual, wherein increased frequency of methylation at the one or more CpG sites compared to reference value ranges for the frequency of methylation at the one or more CpG sites in said at least one biomarker gene selected from the group consisting of RARB and RBP1 in cfDNA from a control subject indicate that the individual will benefit from treatment with the abiraterone acetate. A therapeutically effective amount of the abiraterone acetate may be administered to the individual, if the frequency of methylation at the one or more CpG sites in said at least one biomarker gene selected from the group consisting of RARB and RBP1 indicates that the individual will benefit from treatment with the abiraterone acetate (in some cases, the individual is treated with the abiraterone acetate in combination with docetaxel and/or a corticosteroid). In certain embodiments, the one or more CpG sites in the RARB and RBP1 genes of the cfDNA are selected from cg02499249, cg26124016, cg12479047, cg20899354, cg18094781, cg06720425, and cg15229124. In some embodiments, the frequency of methylation is measured at all the cg02499249, cg26124016, cg12479047, cg20899354, cg18094781, cg06720425, and cg15229124 CpG sites in the cfDNA.

In some embodiments, the methylated cfDNA biomarkers are used for monitoring for a recurrence of prostate cancer in a patient. For example, a first cfDNA can be obtained from the patient after treatment for a previous occurrence of prostate cancer at a first time point when the patient is characterized as cancer-free from imaging or other diagnostic modalities. The levels or frequency of methylation at one or more CpG sites in promoter regions of one or more biomarker genes in cfDNA from the first cfDNA sample can be measured, wherein the one or more biomarker genes are selected from APC, CD44, PYCARD, RARB, and RBP1. A second cfDNA sample can be obtained from the patient at a second time point during a period of monitoring for the recurrence. The levels or frequency of methylation at one or more CpG sites in promoter regions of one or more biomarker genes in cfDNA from the second cfDNA sample can also be measured, wherein the one or more biomarker genes are selected from APC, CD44, PYCARD, RARB, and RBP1, wherein increased levels or frequency of methylation at the one or more CpG sites in the promoter regions of the one or more biomarker genes selected from APC, CD44, PYCARD, RARB, and RBP1 in the cfDNA of the second cfDNA sample compared to the cfDNA of the first cfDNA sample indicate that the prostate cancer has recurred. If the patient has a positive diagnosis for the recurrence of the prostate cancer based on the levels or frequency of methylation of the one or more CpG sites, the patient should be treated for the recurrence of the prostate cancer. In some embodiments, the patient is monitored for a recurrence over a period of time by repeatedly collecting cfDNA samples at intervals and analyzing the cfDNA to determine whether or not the patient has a recurrence of prostate cancer. In some embodiments, the patient is monitored for a recurrence repeatedly over a period of 1 month, 2 months, 4 months, 6 months, 8 month, 1 year, 2 years, 3 years, 4 years, 5 years, or longer by the methods described herein.

In some embodiments, the subject methods include providing an analysis indicating whether the individual is determined to have prostate cancer or a recurrence of prostate cancer. The analysis may further provide an analysis of whether an individual is responsive or not responsive to a treatment, or whether the individual is determined to be maintaining responsiveness or not maintaining responsiveness to a treatment for prostate cancer. As described above, an analysis can be an oral or written report (e.g., written or electronic document). The analysis can be provided to the subject, to the subject's physician, to a testing facility, etc. The analysis can also be accessible as a website address via the internet. In some such cases, the analysis can be accessible by multiple different entities (e.g., the subject, the subject's physician, a testing facility, etc.).

Detecting Methylation of cfDNA

Any suitable method known in the art can be used for detecting methylation at CpG sites in cfDNA. Exemplary techniques for detecting methylation include, without limitation, methylation-sensitive arbitrarily-primed polymerase chain reaction (MS AP-PCR), methylation-sensitive single nucleotide primer extension (Ms-SNuPE), methylation-specific PCR (MSP), methylation-sensitive DNA restriction enzyme analysis, restriction enzyme-based sequencing, restriction enzyme-based microarray analysis, combined bisulfite restriction analysis (COBRA), methylated CpG island amplification (MCA), methylated CpG island amplification and microarray (MCAM), HpaII tiny fragment enrichment by ligation-mediated PCR (HELP), bisulfite sequencing, bisulfite microarray analysis, methylation-specific pyrosequencing, HELP-sequencing (HELP-seq), TET-assisted pyridine borane sequencing (TAPS), GlaI hydrolysis and ligation adapter dependent PCR (GLAD-PCR), methylated DNA immunoprecipitation-sequencing (MeDIP-Seq), or methylated DNA immunoprecipitation-microarray analysis (MeDI P-chip), Southern blotting with methyl-sensitive restriction enzymes, and methylation-specific giant magnetoresistive sensor-based microarray analysis.

Bisulfite sequencing uses bisulfite treatment of DNA before sequencing to detect methylation sites. Treatment of DNA with bisulfite converts cytosine residues to uracil, but does not affect methylated cytosine residues. After bisulfite treatment, the only cytosines remaining in the DNA are methylated cytosines. Thus, sequencing the DNA after bisulfite treatment reveals the methylation status of individual cytosine residues at single-nucleotide resolution (Reinders et al. (2010) Epigenomics 2(2):209-20, Chatterjee et al. (2012) Nucleic Acids Research 40(10): e79, Wreczycka et al. (2017) J. Biotechnol. 261:105-115, Shafi et al. (2018) Brief Bioinform. 19(5):737-753; herein incorporated by reference).

The MS AP-PCR assay uses methylation-sensitive restriction enzymes to digest DNA and PCR with CG-rich primers to selectively amplify regions that contain CpG dinucleotides (see, e.g., Gonzalgo et al. (1997) Cancer Research 57: 594-599, herein incorporated by reference).

The MethyLight assay uses bisulfite-dependent, quantitative fluorescence-based real-time PCR with methylation-specific priming and methylation-specific fluorescent probing for detection of DNA methylation. Digital MethyLight combines the MethyLight assay with digital PCR to allow detection of individual methylated molecules (see, e.g., Eads et al. (1999) Cancer Res. 59: 2302-2306, Campan et al. (2018) Methods Mol. Biol. 1708:497-513; herein incorporated by reference).

The HeavyMethy assay uses methylation specific blocking probes (also referred to herein as blockers) covering CpG positions between, or covered by, amplification primers to enable methylation-specific selective amplification of a nucleic acid sample.

The HeavyMethyl MethyLight assay is a variation of the MethyLight™ assay, wherein the MethyLight™ assay is combined with methylation specific blocking probes covering CpG positions between the amplification primers.

The Ms-SNuPE assay uses bisulfite treatment of DNA combined with PCR using primers designed to hybridize immediately upstream of the CpG site(s) and electrophoresis of amplicons on polyacrylamide gels for visualization and quantitation. Treatment of DNA (genomic or cfDNA) with sodium bisulfite causes unmethylated cytosines to be converted to uracils, During the PCR step, uracil is replicated as thymine, and methylcytosine is replicated as cytosine during amplification. The ratio of methylated versus unmethylated cytosine (C versus T) at the original CpG sites can be determined by incubating the gel-isolated PCR product, primer(s), and Taq polymerase with either [³²P]dCTP or [³²P]TTP followed by denaturing polyacrylamide gel electrophoresis and phosphorimage analysis. Ms-SNuPE primers can also be designed to incorporate either [³²P]dATP or [³²P]dGTP into the opposite strand to assess methylation status depending on which CpG site is analyzed (see, e.g., Gonzalgo & Jones (1997) Nucleic Acids Res. 25: 2529-2531; Gonzalgo et al. (2007) Nat. Protoc. 2(8):1931-6; herein incorporated by reference).

The MSP assay uses bisulfite treatment of DNA for conversion of non-methylated cytosines to uracils and subsequent amplification with primers specific for methylated versus unmethylated DNA (see, e.g., Herman et al. (1996) Proc. Natl. Acad. Sci. USA 93: 9821-9826, and U.S. Pat. No. 5,786,146; herein incorporated by reference).

The COBRA assay uses bisulfite treatment of DNA for conversion of non-methylated cytosines to uracils, locus-specific PCR amplification of the bisulfite-converted DNA, restriction digestion, electrophoresis an analysis of restriction patterns on a gel (see, e.g., Xiong & Laird (1997) Nucleic Acids Res. 25: 2532-2534; Bilichak et al. (2017) Methods Mol. Biol. 1456:63-71; herein incorporated by reference).

The MCA assay uses methylation-sensitive restriction enzymes to digest DNA, followed by adaptor ligation and PCR to selectively amplify methylated CpG-rich sequences (see, e.g., Toyota et al. (1999) Cancer Res. 59: 2307-12, and WO 00/26401A1; herein incorporated by reference).

The MCAM assay uses MCA in combination with a CpG island microarray to detect DNA methylation in a high-throughput fashion (see, e.g., Estecio et al. (2007) Genome Res. 17(10):1529-1536; herein incorporated by reference).

The HELP assay uses the methylation-sensitive restriction enzyme, HpaII, to cut DNA, and a methylation-insensitive isoschizomer, MspI, as a control. Microarray analysis is performed with microarrays containing probes designed to detect the HpaII/MspI fragments. HELP-seq combines the HELP assay with massively parallel sequencing of DNA methylation sites (see, e.g., Greally (2018) Methods Mol. Biol. 1708:191-207; Suzuki et al. (2010) Methods 52(3):218-22; herein incorporated by reference).

The GLAD-PCR assay uses a site-specific methyl-directed DNA-endonucleases that cleave only methylated DNA, followed by ligation of DNA fragments to universal adapters for high-throughput PCR (see, e.g., Malyshev et al. (2020) Acta Naturae 12(3):124-133; Russian Patent RU 2525710; herein incorporated by reference).

The MeDIP assay uses an antibody against 5-methylcytosine to immunoprecipitate methylated DNA fragments. This technique can be combined with high-throughput DNA detection methods using microarray hybridization (MeDIP-chip) or next-generation sequencing (MeDIP-seq). See, e.g., Weber et al. (2005) Nat. Genet. 37 (8): 853-862, Palmke et al. (2011) Methods 53(2):175-184, Quackenbush et al. (2008) Cancer Res. 68(6):1786-1796, Zhu et al. (2019) Analyst 144(6):1904-1915, Yang et al. (2014) Life Sci. 113(1-2):45-54; herein incorporated by reference.

TET-assisted pyridine borane sequencing (TAPS) uses the ten-eleven translocation (TET) enzyme to catalyze oxidation of 5-methylcytosine and 5-hydroxymethylcytosine to 5-carboxylcytosine, followed by pyridine borane reduction to produce dihydrouracil. Unmodified cytosine is not affected. See, e.g., Liu et al, (2019) Nat Biotechnol, 37:424-429; herein incorporated by reference.

Methylation-specific giant magnetoresistive sensor-based microarray analysis combines methylation specific PCR and melt curve analysis on a giant magnetoresistive (GMR) biosensor. The GMR biosensor comprises synthetic DNA probes that target methylated or unmethylated CpG sites in the PCR amplicons. After hybridization of the PCR amplicons to the GMR biosensor, the difference in melting temperature (Tm) between the two types of probes is measured. See, e.g., Rizzi et al. (2017) ACS Nano. 11(9): 8864-8870, Nesvet et al. (2019) Biosens Bioelectron 124-125:136-142; herein incorporated by reference.

Southern Blotting can also be used to detect DNA methylation. The DNA is first digested with methylation-sensitive restriction enzymes, and the restriction fragments are analyzed by Southern Blot.

Sequences are said to be “differentially methylated” or as having a “difference in methylation” or having a “different methylation state” when they differ in the extent (e.g., one has increased or decreased methylation relative to the other), frequency, or pattern of methylation. The term “differential methylation” refers to a difference in the level or pattern of nucleic acid methylation in a cancer positive sample as compared with the level or pattern of nucleic acid methylation in a cancer negative sample. It may also refer to the difference in levels or patterns between patients that have recurrence of cancer after surgery versus patients who do not have recurrence.

Methylation status can optionally be represented or indicated by a “methylation value” (e.g., representing a methylation frequency, fraction, ratio, percent, etc.). A methylation value can be generated, for example, by quantifying the amount of intact nucleic acid present following restriction digestion with a methylation dependent restriction enzyme or by comparing amplification profiles after bisulfite reaction or by comparing sequences of bisulfite-treated and untreated nucleic acids. Accordingly, a value, e.g., a methylation value, represents the methylation status and can thus be used as a quantitative indicator of methylation status across multiple copies of a locus. This is of particular use when it is desirable to compare the methylation status of a sequence in a sample to a threshold or reference value.

The methylation state may be expressed in terms of a fraction or percentage of individual strands of DNA that is methylated at a particular site relative to the total population of DNA in the sample comprising that particular site. As used herein, “methylation frequency” or “methylation percent (%)” refer to the number of instances in which a molecule or locus is methylated relative to the number of instances the molecule or locus is unmethylated.

### Data Analysis

In some embodiments, one or more pattern recognition methods can be used in analyzing the data for cfDNA methylation. The quantitative values may be combined in linear or non-linear fashion to calculate one or more risk scores for prostate cancer for an individual. In some embodiments, measurements for a methylated cfDNA biomarker or combinations of biomarkers are formulated into linear or non-linear models or algorithms (e.g., a ‘biomarker signature’) and converted into a likelihood score. This likelihood score indicates the probability that a cfDNA sample is from a patient who has no evidence of disease or a patient who has prostate cancer. A likelihood score can also be used to distinguish among different stages of cancer progression. The models and/or algorithms can be provided in machine readable format, and may be used to correlate the frequency or levels of methylation at CpG sites in cfDNA biomarker genes or a biomarker profile with a disease state, and/or to designate a treatment modality for a patient or class of patients.

Analyzing the levels of a plurality of biomarkers may comprise the use of an algorithm or classifier. In some embodiments, a machine learning algorithm is used to classify a patient as having prostate cancer. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering.

In some instances, the machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing.

Preferably, the machine learning algorithms may include, but are not limited to, Average One-Dependence Estimators (AODE), Fisher's linear discriminant, Logistic regression, Perceptron, Multilayer Perceptron, Artificial Neural Networks, Support vector machines, Quadratic classifiers, Boosting, Decision trees, C4.5, Bayesian networks, Hidden Markov models, High-Dimensional Discriminant Analysis, and Gaussian Mixture Models. The machine learning algorithm may comprise support vector machines, Naïve Bayes classifier, k-nearest neighbor, high-dimensional discriminant analysis, or Gaussian mixture models. In some instances, the machine learning algorithm comprises Random Forests.

### Kits

Also provided are kits that can be used to detect the methylated cfDNA biomarkers described herein. Such kits can be used to diagnose a subject with prostate cancer, detect a recurrence of prostate cancer, therapy selection, or monitoring responses to treatment. The kit may include one or more agents for detection of methylated cfDNA biomarkers, a container for holding a biological sample comprising cfDNA (e.g., blood or plasma) isolated from a human subject suspected of having prostate cancer; and printed instructions for reacting agents with the biological sample or a portion of the biological sample to detect the frequency or level of methylation at one or more CpG sites in cfDNA in the biological sample. The agents may be packaged in separate containers. The kit may further comprise one or more control reference samples and reagents for performing a methylation assay (e.g., bisulfite sequencing, MS AP-PCR, MethyLight™, Digital MethyLight™, HeavyMethyl™, HeavyMethyl™ MethyLight™, Ms-SNuPE, MSP, COBRA, MCA, MCAM, HELP, HELP-seq, GLAD-PCR, MeDIP-Seq, MeDIP-chip, and the like). For example, the subject kits may include agents for determining the frequency or level of methylation such as a bisulfite reagent, methylation-sensitive restriction enzymes, PCR primers that selectively amplify DNA regions that contain CpG dinucleotides, methylation-specific primers, methylation-specific probes, or a combination thereof.

For example, the kits can be used to detect methylation of one or more of the biomarkers described herein, which show increased frequency of methylation in cfDNA samples from patients who have prostate cancer compared to healthy control subjects or subjects without cancer. In some embodiments, a kit comprises agents for determining the frequency or level of methylation at one or more CpG sites in the genes: APC, CD44, PYCARD, RARB, and RBP1. In some embodiments, the kit comprises agents for determining the frequency or level of methylation at one or more CpG sites selected from cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124, and CpG sites located within 200 nucleotides thereof. In some embodiments, the kit comprises agents for determining the frequency or level of methylation at the CpG sites: cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124.

The kit can comprise one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, flash drive, and the like, on which the information has been recorded. Yet another form of these instructions that may be present is a website address which may be used via the internet to access the information at a removed site.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-43 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method of diagnosing and treating prostate cancer in a patient, the method comprising:
a) obtaining a blood sample from the patient;
b) measuring frequency of methylation at one or more CpG sites in at least one biomarker gene selected from the group consisting of APC, CD44, PYCARD, RARB, and RBP1 in cell-free DNA (cfDNA) in the blood sample, wherein increased frequency of methylation at the one or more CpG sites compared to reference value ranges for the frequency of methylation at the one or more CpG sites in said at least one biomarker gene selected from the group consisting of APC, CD44, PYCARD, RARB, and RBP1 in cfDNA from a control subject indicate that the patient has prostate cancer; and
c) treating the patient for the prostate cancer, if the patient has a positive diagnosis for prostate cancer based on the frequency of methylation at the one or more CpG sites.

2. The method of aspect 1, wherein said one or more CpG sites are selected from cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124, and CpG sites located within 200 nucleotides thereof.

3. The method of aspect 2, wherein said measuring frequency of methylation comprising measuring the frequency of methylation at the cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124 CpG sites in the cfDNA.

4. The method of any one of aspects 1 to 3, further comprising calculating a prostate cancer risk score based on the methylation frequency at the CpG sites in the APC, CD44, PYCARD, RARB, and RBP1 genes of the cfDNA using one or more algorithms.

5. The method of any one of aspects 1 to 4, wherein said treating the patient for the prostate cancer comprises performing a surgical resection, a prostatectomy, external beam radiation therapy, particle therapy, brachytherapy, cryosurgery, high-intensity focused ultrasound, androgen deprivation therapy, chemotherapy, targeted therapy, or immunotherapy, or a combination thereof.

6. The method of any one of aspects 1 to 5, wherein said measuring the frequency of methylation comprises performing methylation-sensitive arbitrarily-primed polymerase chain reaction (MS AP-PCR), methylation-sensitive single nucleotide primer extension (Ms-SNuPE), methylation-specific PCR (MSP), methylation-sensitive DNA restriction enzyme analysis, restriction enzyme-based sequencing, restriction enzyme-based microarray analysis, combined bisulfite restriction analysis (COBRA), methylated CpG island amplification (MCA), methylated CpG island amplification and microarray (MCAM), HpaII tiny fragment enrichment by ligation-mediated PCR (HELP), bisulfite sequencing, bisulfite microarray analysis, methylation-specific pyrosequencing, HELP-sequencing (HELP-seq), TET-assisted pyridine borane sequencing (TAPS), GlaI hydrolysis and ligation adapter dependent PCR (GLAD-PCR), methylated DNA immunoprecipitation-sequencing (MeDIP-Seq), or methylated DNA immunoprecipitation-microarray analysis (MeDIP-chip), Southern blotting with methyl-sensitive restriction enzymes, or methylation-specific giant magnetoresistive sensor-based microarray analysis.

7. The method of any one of aspects 1 to 6, further comprising measuring blood levels of prostate-specific antigen (PSA), blood levels of kallikrein 2, or urine levels of prostate cancer antigen 3 (PCA3), or a combination thereof, wherein detection of increased blood levels of PSA, increased blood levels of kallikrein 2, or increased urine levels of PCA3 in combination with increased frequency of methylation at the one or more CpG sites in the one or more genes selected from the group consisting of APC, CD44, PYCARD, RARB, and RBP1 compared to reference value ranges for the blood levels of PSA and kallikrein 2, the urine levels of PCA3, and the frequency of methylation at the one or more CpG sites in the one or more genes selected from the group consisting of APC, CD44, PYCARD, RARB, and RBP1 for a control subject indicate that the patient has a positive diagnosis for the prostate cancer.

8. The method of any one of aspects 1 to 7, wherein the patient has a risk factor that makes the patient susceptible to developing prostate cancer.

9. The method of aspect 8, wherein the risk factor is age over 50, family history, genetic predisposition, environmental exposure to a carcinogen, obesity, a sexually transmitted disease, or prostatitis.

10. A method of monitoring prostate cancer in a patient, the method comprising:
a) obtaining a first blood sample from the patient at a first time point and a second blood sample from the patient later at a second time point; and
b) detecting methylation at one or more CpG sites in one or more genes of circulating free DNA (cfDNA) in the first blood sample and the second blood sample, wherein the one or more genes are selected from the group consisting of APC, CD44, PYCARD, RARB, and RBP1, wherein detection of increased frequency of methylation of the CpG sites in the one or more genes selected from the group consisting of APC, CD44, PYCARD, RARB, and RBP1 in the cfDNA of the second blood sample compared to the cfDNA of the first blood sample indicate that the prostate cancer is progressing, and detection of decreased frequency of methylation of the CpG sites in the one or more genes selected from the group consisting of APC, CD44, PYCARD, RARB, and RBP1 in the cfDNA of the second blood sample compared to the cfDNA of the first blood sample indicate that the prostate cancer is not progressing.

11. The method of aspect 10, wherein the prostate cancer is a primary tumor, a metastasis, or a recurrence.

12. The method of aspect 10 or 11, wherein the first time point is before a treatment of the patient for prostate cancer is started and the second time point is during or after the treatment.

13. The method of aspect 12, wherein the treatment comprises surgical resection, prostatectomy, external beam radiation therapy, particle therapy, brachytherapy, cryosurgery, high-intensity focused ultrasound, androgen deprivation therapy, chemotherapy, targeted therapy, or immunotherapy, or a combination thereof.

14. The method of any one of aspects 10 to 13, further comprising repeating steps a) and b).

15. The method of any one of aspects 10 to 14, further comprising increasing dosage or frequency of a treatment for prostate cancer, changing to a different treatment, or starting palliative care for the patient if the prostate cancer is progressing.

16. The method of any one of aspects 10 to 15, wherein the one or more CpG sites are selected from cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124, and CpG sites located within 200 nucleotides thereof.

17. The method of aspect 16, wherein said detecting methylation comprises measuring frequency of methylation at the cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124 CpG sites in the cfDNA.

18. The method of any one of aspects 10 to 17, further comprising measuring blood levels of prostate-specific antigen (PSA), blood levels of kallikrein 2, or urine levels of prostate cancer antigen 3 (PCA3), or a combination thereof, wherein detection of increased blood levels of PSA, increased blood levels of kallikrein 2, or increased urine levels of PCA3 in combination with increased frequency of methylation at the one or more CpG sites in the one or more genes selected from the group consisting of APC, CD44, PYCARD, RARB, and RBP1 in the second blood sample compared to the first blood sample indicate that the prostate cancer is progressing; and decreased blood levels of prostate-specific antigen (PSA), decreased blood levels of kallikrein 2, or decreased urine levels of prostate cancer antigen 3 (PCA3) in combination with decreased frequency of methylation at the one or more CpG sites in the one or more genes selected from the group consisting of APC, CD44, PYCARD, RARB, and RBP1 in the second blood sample compared to the first blood sample indicate that the prostate cancer is not progressing.

19. The method of any one of aspects 10 to 18, wherein said detecting the methylation of CpG sites in the cfDNA comprises performing methylation-sensitive arbitrarily-primed polymerase chain reaction (MS AP-PCR), methylation-sensitive single nucleotide primer extension (Ms-SNuPE), methylation-specific PCR (MSP), methylation-sensitive DNA restriction enzyme analysis, restriction enzyme-based sequencing, restriction enzyme-based microarray analysis, combined bisulfite restriction analysis (COBRA), methylated CpG island amplification (MCA), methylated CpG island amplification and microarray (MCAM), HpaII tiny fragment enrichment by ligation-mediated PCR (HELP), bisulfite sequencing, bisulfite microarray analysis, methylation-specific pyrosequencing, HELP-sequencing (HELP-seq), TET-assisted pyridine borane sequencing (TAPS), GlaI hydrolysis and ligation adapter dependent PCR (GLAD-PCR), methylated DNA immunoprecipitation-sequencing (MeDIP-Seq), or methylated DNA immunoprecipitation-microarray analysis (MeDIP-chip), Southern blotting with methyl-sensitive restriction enzymes, or methylation-specific giant magnetoresistive sensor-based microarray analysis.

20. A method of monitoring for a recurrence of prostate cancer in a patient, the method comprising:
a) obtaining a first circulating free DNA (cfDNA) sample from the patient after treatment for a previous occurrence of prostate cancer at a first time point when the patient is characterized as cancer-free from imaging or other diagnostic modalities;
b) detecting methylation at one or more CpG sites of one or more biomarker genes in cfDNA from the first cfDNA sample, wherein the one or more biomarker genes are selected from APC, CD44, PYCARD, RARB, and RBP1;
c) obtaining a second cfDNA sample from the patient at a second time point during a period of monitoring for the recurrence;
d) detecting methylation at the one or more CpG sites of the one or more biomarker genes in cfDNA from the second cfDNA sample, wherein the one or more biomarker genes are selected from APC, CD44, PYCARD, RARB, and RBP1, wherein increased frequency of methylation at the one or more CpG sites of the one or more biomarker genes selected from APC, CD44, PYCARD, RARB, and RBP1 in the cfDNA of the second cfDNA sample compared to the cfDNA of the first cfDNA sample indicates that the prostate cancer has recurred; and
e) repeating steps c)-e) subsequently during the period of monitoring for the recurrence.

21. The method of aspect 20, further comprising treating the patient for the recurrence of the prostate cancer, if the patient has a positive diagnosis for the recurrence of the prostate cancer based on the levels of methylation of the one or more CpG sites.

22. The method of aspect 20 or 21, wherein said treating the patient for the recurrence of prostate cancer comprises surgical resection, prostatectomy, external beam radiation therapy, particle therapy, brachytherapy, cryosurgery, high-intensity focused ultrasound, androgen deprivation therapy, chemotherapy, targeted therapy, or immunotherapy, or a combination thereof.

23. The method of any one of aspects 20 to 22, wherein the one or more CpG sites are selected from cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124, and CpG sites located within 200 nucleotides thereof.

24. The method of aspect 23, wherein said detecting methylation comprises measuring frequency of methylation at the cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124 CpG sites in the cfDNA.

25. The method of any one of aspects 20 to 24, further comprising measuring blood levels of prostate-specific antigen (PSA), blood levels of kallikrein 2, or urine levels of prostate cancer antigen 3 (PCA3), or a combination thereof for the patient, wherein increased blood levels of PSA, increased blood levels of kallikrein 2, or increased urine levels of PCA3 in combination with increased frequency of methylation at the one or more CpG sites in the one or more genes selected from the group consisting APC, CD44, PYCARD, RARB, and RBP1 in the cfDNA from the patient compared to reference value ranges for the blood levels of PSA, the blood levels of kallikrein 2, the urine levels of PCA3, and the frequency of methylation at the one or more CpG sites in the one or more genes selected from the group consisting of APC, CD44, PYCARD, RARB, and RBP1 indicate that the patient has a positive diagnosis for the recurrence of prostate cancer.

26. The method of any one of aspects 20 to 25, wherein the cfDNA sample is a blood sample or plasma sample comprising cfDNA.

27. The method of any one of aspects 20 to 26, wherein said detecting the methylation of CpG sites in the cfDNA comprises performing methylation-sensitive arbitrarily-primed polymerase chain reaction (MS AP-PCR), methylation-sensitive single nucleotide primer extension (Ms-SNuPE), methylation-specific PCR (MSP), methylation-sensitive DNA restriction enzyme analysis, restriction enzyme-based sequencing, restriction enzyme-based microarray analysis, combined bisulfite restriction analysis (COBRA), methylated CpG island amplification (MCA), methylated CpG island amplification and microarray (MCAM), HpaII tiny fragment enrichment by ligation-mediated PCR (HELP), bisulfite sequencing, bisulfite microarray analysis, methylation-specific pyrosequencing, HELP-sequencing (HELP-seq), TET-assisted pyridine borane sequencing (TAPS), GlaI hydrolysis and ligation adapter dependent PCR (GLAD-PCR), methylated DNA immunoprecipitation-sequencing (MeDIP-Seq), or methylated DNA immunoprecipitation-microarray analysis (MeDIP-chip), Southern blotting with methyl-sensitive restriction enzymes, or methylation-specific giant magnetoresistive sensor-based microarray analysis.

28. A method for selecting an individual with prostate cancer for treatment with abiraterone acetate (17-(3-pyridinyl)androsta-5,16-dien-3β-ol acetate) and treating the individual, the method comprising:
a) obtaining a blood sample from the individual;
b) measuring frequency of methylation at one or more CpG sites in at least one biomarker gene selected from the group consisting of RARB and RBP1 in cell-free DNA (cfDNA) in the blood sample, wherein increased frequency of methylation at the one or more CpG sites compared to reference value ranges for the frequency of methylation at the one or more CpG sites in said at least one biomarker gene selected from the group consisting of RARB and RBP1 in cfDNA from a control subject indicate that the individual will benefit from treatment with the abiraterone acetate; and
c) administering a therapeutically effective amount of the abiraterone acetate to the individual, if the frequency of methylation at the one or more CpG sites in said at least one biomarker gene selected from the group consisting of RARB and RBP1 indicates that the individual will benefit from treatment with the abiraterone acetate.

29. The method of aspect 28, wherein said one or more CpG sites are selected from cg02499249, cg26124016, cg12479047, cg20899354, cg18094781, cg06720425, and cg15229124.

30. The method of aspect 29, wherein said measuring frequency of methylation comprises measuring the frequency of methylation at the cg02499249, cg26124016, cg12479047, cg20899354, cg18094781, cg06720425, and cg15229124 CpG sites in the cfDNA.

31. The method of any one of aspects 28 to 30, wherein the individual is treated with the abiraterone acetate in combination with docetaxel or a corticosteroid.

32. A kit comprising agents for detecting methylation of one or more CpG sites in APC, CD44, PYCARD, RARB, and RBP1 genes in cfDNA.

33. The kit of aspect 32, wherein said one or more CpG sites are selected from cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124, and CpG sites located within 200 nucleotides thereof.

34. The kit of aspect 33, wherein said CpG sites comprise cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124.

35. The kit of any one of aspects 32 to 34, further comprising agents for performing methylation-sensitive arbitrarily-primed polymerase chain reaction (MS AP-PCR), methylation-sensitive single nucleotide primer extension (Ms-SNuPE), methylation-specific PCR (MSP), methylation-sensitive DNA restriction enzyme analysis, restriction enzyme-based sequencing, restriction enzyme-based microarray analysis, combined bisulfite restriction analysis (COBRA), methylated CpG island amplification (MCA), methylated CpG island amplification and microarray (MCAM), HpaII tiny fragment enrichment by ligation-mediated PCR (HELP), bisulfite sequencing, bisulfite microarray analysis, methylation-specific pyrosequencing, HELP-sequencing (HELP-seq), TET-assisted pyridine borane sequencing (TAPS), GlaI hydrolysis and ligation adapter dependent PCR (GLAD-PCR), methylated DNA immunoprecipitation-sequencing (MeDIP-Seq), or methylated DNA immunoprecipitation-microarray analysis (MeDI P-chip), Southern blotting with methyl-sensitive restriction enzymes, or methylation-specific giant magnetoresistive sensor-based microarray analysis.

36. The kit of any one of aspects 32 to 35, wherein said agents comprise a bisulfite reagent, methylation-sensitive restriction enzymes, PCR primers that selectively amplify DNA regions that contain CpG dinucleotides, methylation-specific primers, methylation-specific probes, or a combination thereof.

37. The kit of any one of aspects 32 to 36, further comprising reagents for measuring PSA, kallikrein 2, or PCA3, or a combination thereof.

38. The kit of any one of aspects 32 to 37, further comprising instructions for using the kit for diagnosis of prostate cancer, detecting recurrence of prostate cancer, or monitoring treatment of prostate cancer.

39. A cell-free DNA hypermethylated at one or more CpG sites in at least one biomarker gene selected from APC, CD44, PYCARD, RARB, and RBP1 for use in diagnosing prostate cancer, detecting recurrence of prostate cancer, or monitoring treatment of prostate cancer.

40 The cell-free DNA of aspect 39, wherein said one or more CpG sites are selected from cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124, and CpG sites located within 200 nucleotides thereof.

41. An in vitro method of diagnosing prostate cancer in a patient, the method comprising:
a) obtaining a blood sample from the patient; and
b) measuring frequency of methylation at one or more CpG sites in at least one biomarker gene selected from the group consisting of APC, CD44, PYCARD, RARB, and RBP1 in cell-free DNA (cfDNA) in the blood sample, wherein increased frequency of methylation at the one or more CpG sites compared to reference value ranges for the frequency of methylation at the one or more CpG sites in said at least one biomarker gene selected from the group consisting of APC, CD44, PYCARD, RARB, and RBP1 in cfDNA from a control subject indicate that the patient has prostate cancer.

42. The in vitro method of aspect 41, wherein said one or more CpG sites are selected from cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124, and CpG sites located within 200 nucleotides thereof.

43. The method of aspect 42, wherein said measuring frequency of methylation comprises measuring the frequency of methylation at the cg02499249, cg26124016, cg12479047, cg20899354, cg18094781, cg06720425, and cg15229124 CpG sites in the cfDNA.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

### Example 1

### Cell-Free DNA Biomarker Panel and Assay for Diagnosis, Therapy Response Monitoring and Therapy Selection for Prostate Cancer Patients

### Introduction

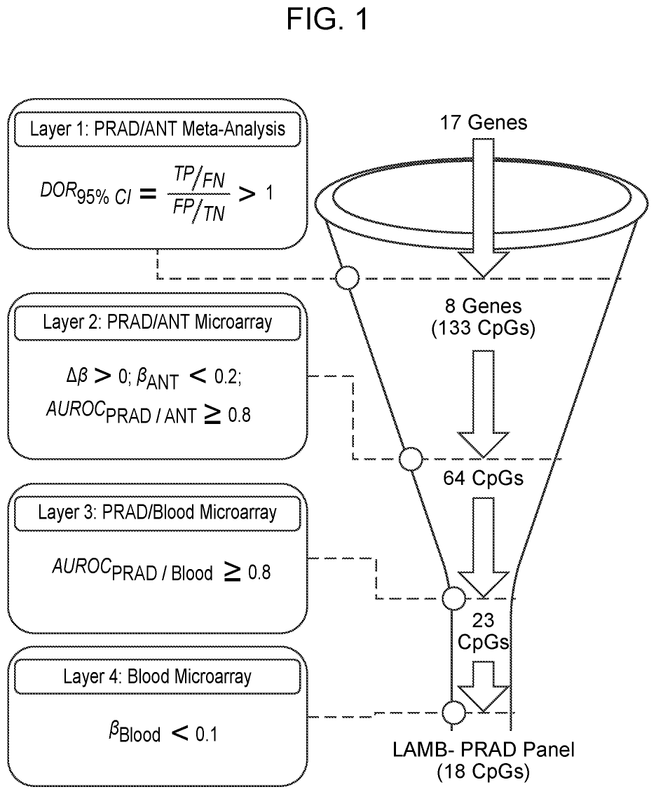

Blood-based biomarkers for cancer screening, therapy response monitoring, and therapy selection are an active area of interest with directly translatable implications for clinical care. Of these biomarker types, methylated cell-free DNA (cfDNA) has shown promise for the screening of liver and colorectal cancer in general populations¹⁻². Cancer development has been linked to the functional silencing of tumor suppressor genes through the hypermethylation of CpG dinucleotides in their promoter regions. However, these methylated cfDNA biomarkers have been discovered through statistical analyses of samples from relatively small cohorts. Moreover, to potentially guard against false positives from the small cohort sizes, these analyses likely did not account for the biological relevance of the proposed biomarkers. These limitations may cause such methods to identify biomarkers with lower predictive power in other patient cohorts. To develop robust methylated cfDNA panels, we created a biologically-inspired biomarker discovery pipeline, Layered Analysis for Methylated Biomarkers (LAMB), that integrates methylation data from cancer patients and healthy controls to screen tumor suppressor genes for biologically-rooted methylated cfDNA biomarkers (FIG. 1, Table 1). To evaluate these markers for therapy response and selection, we turned to prostate cancer.

During the treatment of prostate cancer patients, who have ADT and CRPC, with AA, serum PSA levels oftentimes are used to assess therapy response and monitor patients⁶. Using PSA for CRPC therapy monitoring has been recommended by multiple urology agencies⁷⁻¹¹. Accordingly, to test if LAMB panels can be used for therapy response monitoring and therapy selection, we performed LAMB on prostate cancer data.

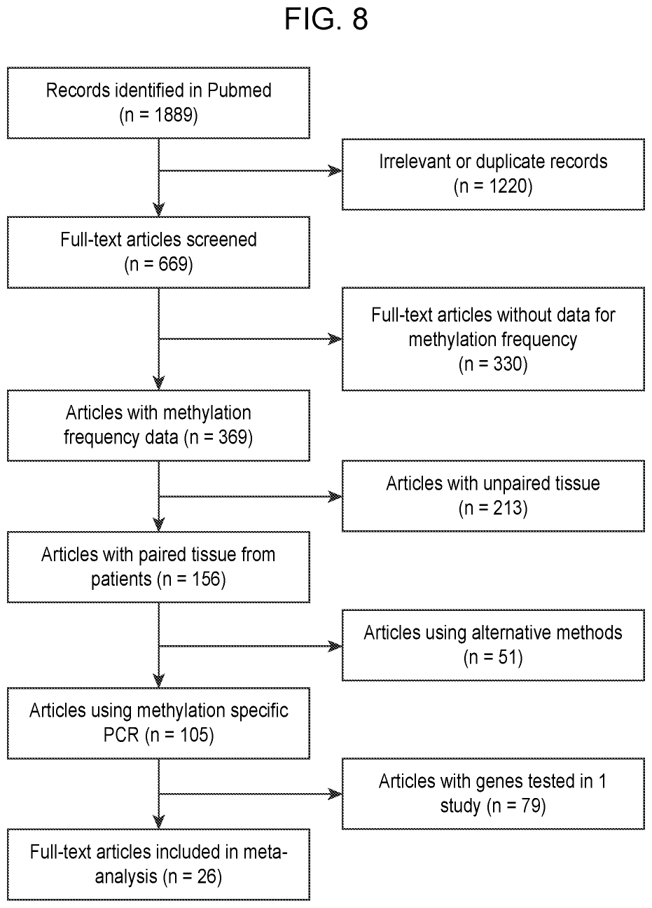

LAMB identifies hypermethylated tumor suppressors in tissues through meta-analysis data and differentially methylated CpGs in the promoters of these tumor suppressors with methylation microarray data. LAMB Layer 1 finds candidate genes by using count data of the number of paired PRADs and adjacent noncancerous tissues (ANTs) hypermethylated for 17 tested genes from 26 studies (FIG. 1, FIG. 8). A random-effects meta-analysis calculated diagnostic odds ratios (DORs) for genes in two or more studies, identifying 8 tumor suppressors for analysis in LAMB Layer 2 (Table 2).

LAMB Layer 2 filters promoter CpGs in candidate tumor suppressors by their methylation frequencies (3). Illumina HumanMethylation450 (450K) microarray data for CpGs in the promoter regions of the 8 tumor suppressor candidate genes were analyzed in paired PRAD and ANT of 145 patients from the Cancer Genome Atlas (TCGA) and four other studies¹²⁻¹⁵. CpGs with lower mean methylation in PRADs than ANTs, hypermethylation in ANTs, or low predictive power between PRADs and ANTs in a univariate logistic regression model were removed (FIG. 1).

CpGs that survived these criteria were analyzed in LAMB Layer 3. The majority of cfDNA comes from hematopoietic cell death with hepatocytes accounting for ˜1-10% of cfDNA in healthy controls¹³⁻¹⁴. To select methylated cfDNA biomarkers distinguishable from hematopoietic cfDNA, LAMB Layers 3 and 4 screen CpGs against 1722 lysed whole blood samples from healthy control patients. 450K blood data were combined from five studies¹⁵⁻¹⁹. 315 TCGA HCCs were matched to 315 lysed whole blood samples selected from the 1722 blood samples by age, gender, and race. These matched tissue and blood samples were selected without any bias from other demographic, clinical, or methylation frequency factors. LAMB Layer 3 filters out CpG sites with low predictive power between the matched 315 HCC tissues and 315 healthy control blood in a univariate logistic regression model (FIG. 1). To select biomarkers with low background methylation, LAMB Layer 4 filters CpGs solely by their methylation frequency in the remaining 1407 lysed whole blood samples (FIG. 1).

Through its 4 distinct filtering layers, the LAMB pipeline identified a methylated cfDNA biomarker panel (LAMB-PRAD) of 18 CpGs from 5 tumor suppressors (Table 3). The LAMB-PRAD panel was then evaluated on an independent 450K validation dataset of cfDNA from 23 CRPC patients treated with AA and docetaxel¹⁶. The dataset contained technical replicates for some of the patient visits; methylation data for patient visits with technical replicates whose coefficient of variation was larger than 20% were discarded, and only patients with baseline, pre-treatment samples were analyzed. As per clinical standards, patients with a >30% decrease in PSA from their baseline blood sample to their first visit sample were classified as responders with all other patients classified as non-responders¹⁷⁻²³. Accordingly, there were 8 responders and 15 non-responders.

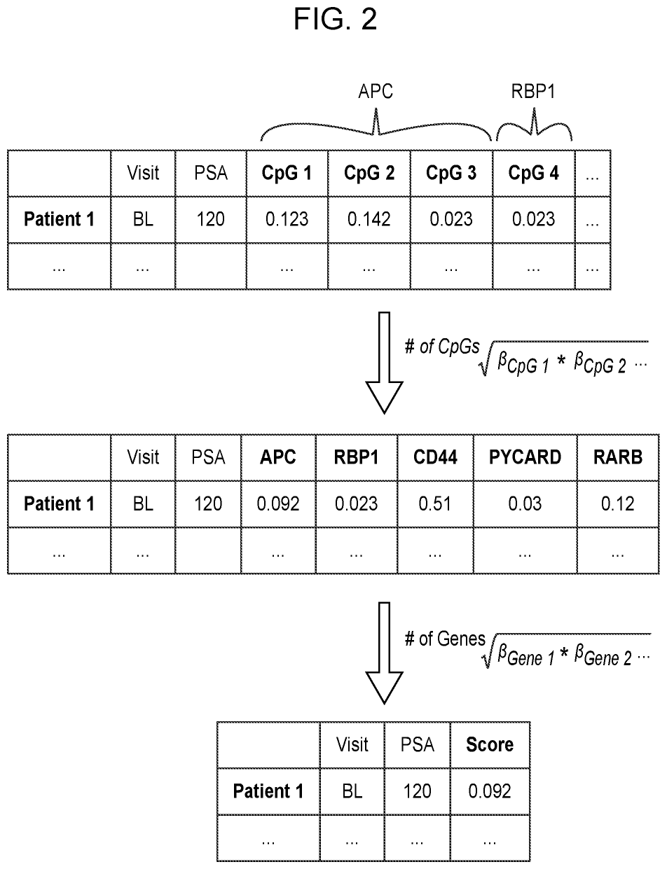

We viewed the LAMB-PRAD panel as 18 CpGs that captured the methylation profile of 5 genes in the cfDNA. We mapped the geometric mean frequency of LAMB-PRAD CpGs in the promoter of a gene to that gene, resulting in 5-gene methylation frequency profiles for the 77 visits across the 23 CRPC patients. The geometric mean normalizes input CpG methylation frequencies so that no CpG dominates weighting of the output gene methylation score. To test the LAMB-PRAD panel on the validation data in an unbiased manner, we calculated geometric mean scores for each visit through the 5-gene methylation frequency profiles (FIG. 2).

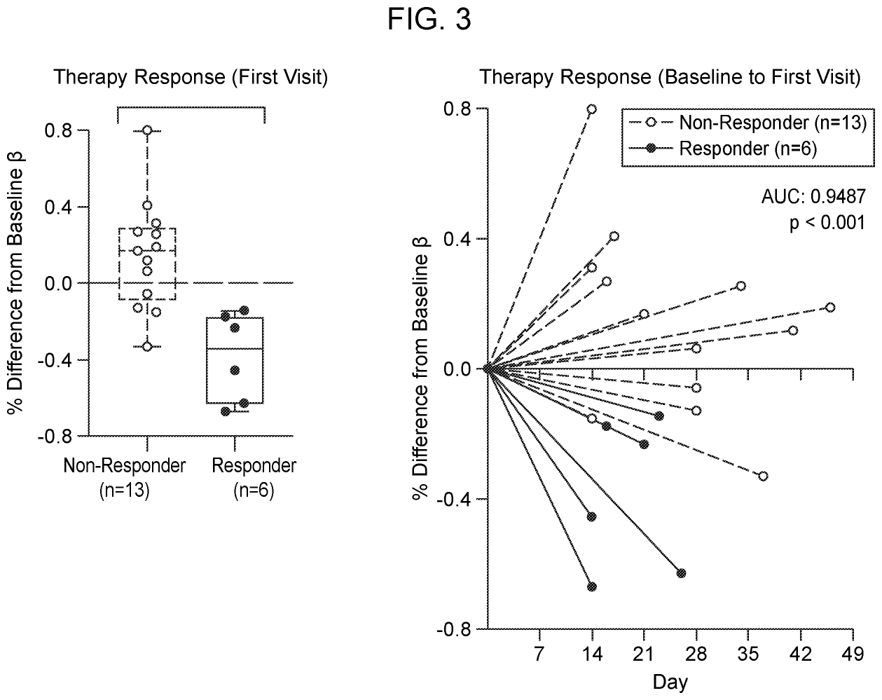

We first tested if differences in LAMB-PRAD methylation scores from cfDNA samples taken from the patients' first visit compared to their baseline scores differentiate responders from non-responders. This metric differentiated responders from non-responders (p<0.001, AUROC: 0.9487, FIG. 3).

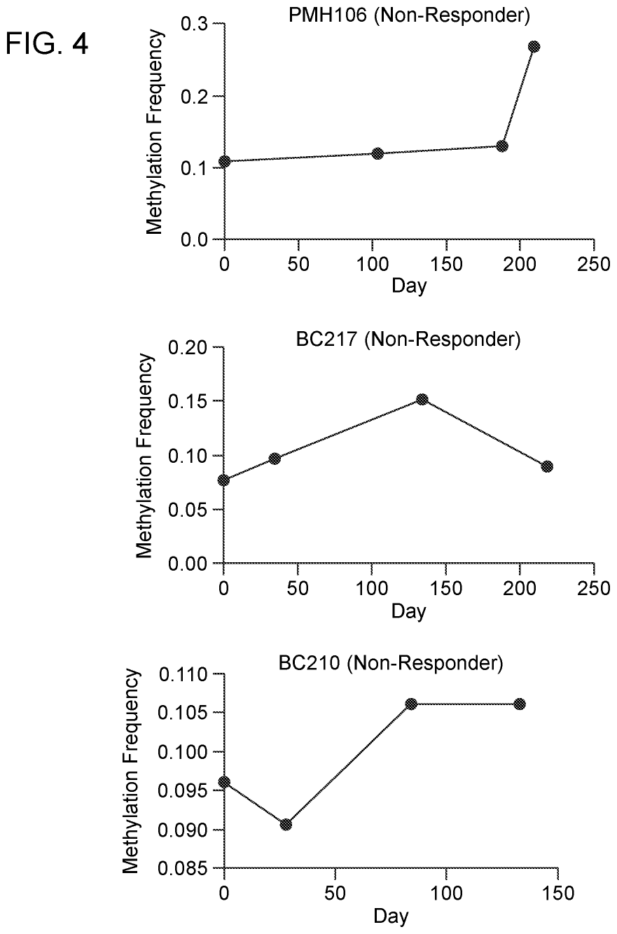

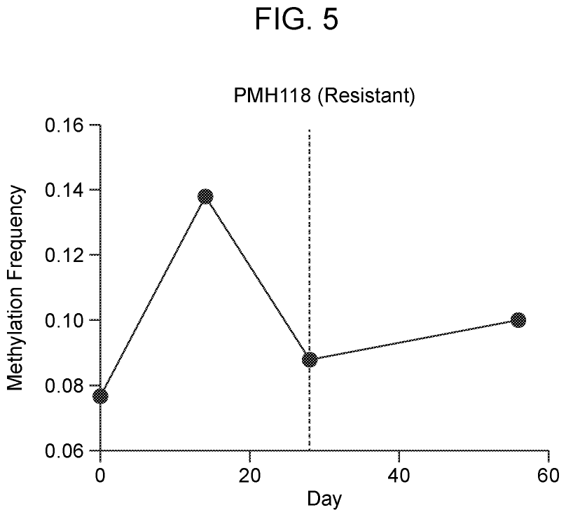

We then examined if LAMB-PRAD scores could be used to monitor tumor progression over the course of therapy. As mentioned before, non-responders were categorized as patients with a <30% decrease in PSA from their baseline blood sample to their first visit sample. Accordingly, these patients will include those with a modest response to AA, those with stable disease, and those with progressive disease. In fact, all but one of the non-responders showed an increase in their LAMB-PRAD score from their baseline or first visit (FIG. 4). Only one of the non-responders (PMH118) showed a decrease in PSA during the course of their treatment. Their LAMB-PRAD score also decreased in that visit (FIG. 5).

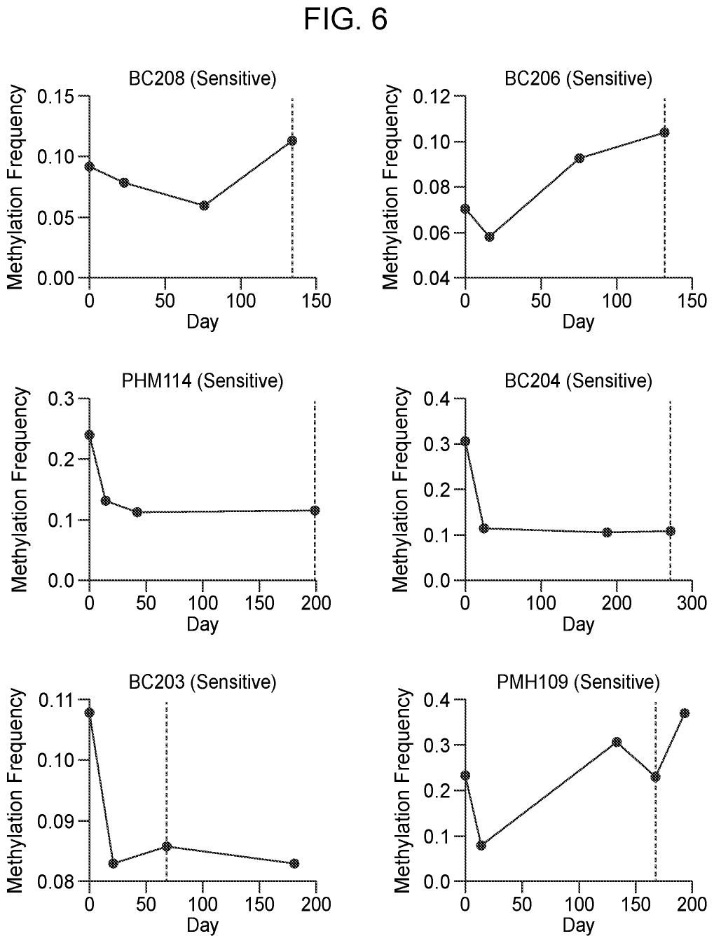

On the other hand, six of the responders showed resistance to AA during the course of their treatment (defined as an increase in PSA from the previous visit). Four patients (BC203, BC206, BC208, PMH109) show sharp increases in LAMB-PRAD score during these visits when compared to the nadir, while the other two patients (PMH114, BC204) show a very slight increase (FIG. 6). These results showcase LAMB-PRAD's potential for monitoring tumor response or resistance over the course of treatment.

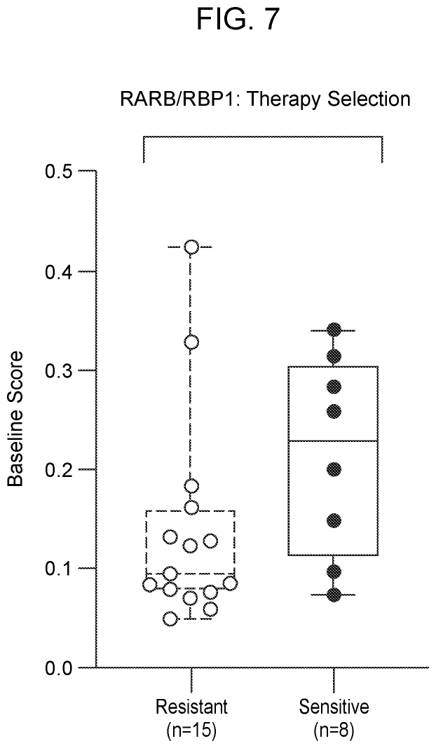

We finally examined the LAMB-PRAD panel to identify therapy selection biomarkers. Typically, these biomarkers require a mechanistic connection tied to the molecular pathways that the therapy affects. In this case, Abiraterone acetate suppresses the overexpression of oncogenes mediated by the overproduction of androgen by directly inhibiting CYP17A1 and lowering testosterone production. Out of the genes in the LAMB-PRAD panel, RARB (Retinoic Acid Receptor Beta) and RBP1 (Retinol Binding Protein 1) directly connect to this molecular pathway through their roles with retinoic acid, which inhibits the 5α-reductase enzyme that metabolizes androgee²⁴⁻²⁸. We found that a combined RARB/RBP1 methylation score (calculated through geometric mean) predicted therapy response in baseline, pre-treatment blood samples, highlighting its potential as a biomarker panel for Abiraterone acetate therapy selection (FIG. 7).

We created the LAMB method with the goal of mining two rich methylation data sources, published studies and 450K data, to discover population-wide methylated cfDNA biomarkers. Using tissue studies identified tumor suppressor genes shown to be hypermethylated in prostate cancer. By incorporating this information into 450K data in a biologically-guided manner, LAMB tests biomarkers across multiple data types, sample types, and patient cohorts in an effort to recapitulate the epigenetic diversity of tumors and patients. Selectively collecting methylation count and frequency data from paired PRAD/ANT and matching tumors to blood by demographic background provides the balance of cases and controls needed to utilize DORs and AUROCs as unbiased metrics to screen biomarkers. As a result, LAMB identified a methylated cfDNA panel for PRAD in CPRC patients that showed high predictive power for therapy response and therapy selection in an external validation dataset. Additional validation of the panel is needed for eventual clinical adoption. Nevertheless, the LAMB method and these validation results could fuel the creation of other physiologically-inspired data mining approaches to identify population-wide, biologically-rooted biomarkers for other diseases beyond liver cancer.

### Methods

### LAMB Layer 1: Gene Meta-Analysis of Tissue Methylation Studies

R.P. independently conducted the screening of abstracts/papers and collected hypermethylation count data from the resulting papers. Differences in included papers and counts were discussed and all three agreed to final decisions. Searches for “((((prostate adenocarcinoma) OR prostate cancer) OR prostate carcinoma) OR PRAD) AND methylation” in PubMed led to 1889 records (as of Jan. 1, 2020). These records were screened for relevance to PRAD tissue methylation, resulting in 669 pertinent tissue methylation papers (FIG. 8). Papers were screened for methylation frequency data, reducing inclusion to 369 studies. The remaining papers were screened for testing PRADs and ANTs from the same patients and use of methylation-specific PCR, further reducing inclusion to 105 papers. Count data was collated for the 26 papers with hypermethylated promoters for paired PRADs and ANTs regarded as true positives and false positives, respectively. Using the “metafor” library in R, a random-effects meta-analysis using the Hartung-Knapp-Sidik-Jonkman method was conducted on genes tested in two or more studies, resulting in adjusted natural logs of DORs (diagnostic odds ratios) with 95% confidence intervals (Table 2)²⁷. Genes with 95% natural log DOR confidence intervals less than 0 were discarded.

### LAMB Layer 2: Promoter CpG Methylation Analysis in PRAD and ANT Microarrays

IIlumina HumanMethylation450 (450K) methylation frequency data was downloaded from the Cancer Genome Atlas (TCGA-PRAD)¹⁰. Recurrent tumors were removed and data for 50 patients with paired primary PRADs and ANTs were used. 450K data for 15 patients (GSE112047), 16 patients (GSE55598), 12 patients (GSE73549), and 52 patients (GSE76938) with paired tissues were downloaded from GEO. These datasets were selected because they had public demographic information, which can be found in their associated studies. All five datasets were combined into a single dataset, and any CpG that was missing in a sample was removed, leading to 229,815 CpGs for 145 patients. The data was logit-transformed, the “ComBat” package in R was utilized to correct for batch effects among the datasets, and the data were inverse logit-transformed²⁸. The remaining CpGs were coupled to genes, features, and chromosomal locations through IIlumina's 450K annotation file. Methylation frequency data for the CpGs in the TSS1500 and TSS200 of the 8 genes from the meta-analysis was extracted for all 145 patients. With the R “pROC” library, mean PRAD methylation, mean ANT methylation, and univariate CpG AUROCs between PRADs and ANTs were calculated for the 145 patients (Table 3)²⁹. CpGs with a lower mean methylation in PRAD than ANT, relative hypermethylation in ANT (methylation frequency: β>0.2), or an AUROC less than 0.8 in a univariate logistic regression model were removed.

### LAMB Layers 3/4: Promoter CpG Methylation Analysis in Whole Blood of Healthy Controls

450K methylation frequency data of lysed whole blood were downloaded for 305, 127, 622, 272, 236, and 160 healthy control patients from GEO (GSE84727, GSE80417, GSE40279, GSE72773, GSE111629, GSE53740)¹⁵⁻¹⁹. The data were combined into a methylation frequency matrix and logit-transformed. The datasets analyzed lysed whole blood and were structured such that healthy controls were identifiable. The data were corrected for batch effects with the “ComBat” package in Python. Methylation frequency data for CpGs identified in LAMB Layer 2 was extracted from the blood data. To perform a differential methylation analysis of HCC tissue to blood, 450K data for 453 separate TCGA PRADs was downloaded. 315 of these tumors were matched to 315 of the whole blood samples from the healthy control patients based on patient age, gender, and race. The ratio of matched blood samples by their GEO dataset mirrored the ratio of total blood samples by their GEO dataset. CpGs with an AUROC <0.8 between PRAD tissue and control blood in a univariate logistic regression model were removed (Table 3). Mean methylation frequencies of remaining CpGs in the remaining 1407 blood samples were calculated. CpGs with a methylation frequency less than 0.1 were identified as LAMB CpGs (Table 3).

### External Validation: LAMB-PRAD Panel Analysis in Cell-Free DNA

450K methylation data was downloaded for all the visits of 23 castration-resistant prostate cancer (CRPC) patients treated with Abiraterone acetate and docetaxel (GSE108462)⁶. As of April 2020, GSE108462 is the only methylation frequency data available for cell-free DNA for CRPC patients. Patients were classified as being responders if their serum PSA levels decreased by >30% at their first visit; the other 15 patients were classified as non-responders. LAMB CpG data were extracted from all the visits, and the geometric mean frequency of the LAMB CpGs in a gene promoter was mapped to that gene to create 5-gene methylation profiles for all the visits of the 23 patients (FIG. 2B). For each visit, the geometric mean of each of the 5-gene methylation profiles was calculated (FIG. 2C). For visits with multiple technical replicates, their data was removed if the coefficient of variation for the LAMB-PRAD scores for the visits exceeded 20%. LAMB-PRAD scores of visits with technical replicates that remained after this filter were averaged by geometric mean to get a LAMB-PRAD score for each of these visits.

To evaluate the LAMB-PRAD panel's ability to identify a patient's response to therapy, data for 13 non-responder and 6 responder patients with pre-treatment, baseline and post-treatment, first visit LAMB-PRAD scores was used. The percent difference in LAMB-PRAD score was calculated by subtracting the baseline score from the first visit score and then dividing the difference by the baseline score. A box-and-whisker plot was created, and both Mann-Whitney and AUC analysis were performed through Prism.

To evaluate the LAMB-PRAD panel's ability to monitor tumor response to therapy, the LbAMB-PRAD scores for one non-responder (PMH118) and six responders (PMH114, PMH109, BC204, BC208, BC206, BC203) were plotted over time. The second visit in which the non-responder saw a decrease in PSA from the first visit was classified as a response to therapy, while the visits in which the responders saw an increase in PSA from the previous visit were classified as acquired resistance to therapy.

To evaluate the RARB/RBP1 panel's ability to predict therapy response from baseline samples as therapy selection biomarkers, the geometric mean of the two genes' methylation frequencies was calculated for the baseline samples of the 15 non-responders and 8 responders. A box-and-whisker plot was created, and both Mann-Whitney and AUC analysis were performed through Prism.

TABLE 1
Datasets Used in LAMB-PRAD Analysis

| Layer | Dataset | Type (n) | Description |
|---|---|---|---|
| 1: PRAD/ANT Meta- | 26 published | PRAD paired to | Inclusion criteria: written in |
| Analysis | tissue papers | ANT (1476) | English; paired PRAD with |
| | | | ANT samples; methylation- |
| | | | specific PCR; contains genes |
| | | | tested in 1 other paper that |
| | | | met inclusion criteria |
| 2: PRAD/ANT | TCGA-PRAD | PRAD/ANT (50) | Paired primary PRAD/ANT |
| Microarray | GSE 112047 | PRAD/ANT (15) | Paired primary PRAD/ANT |
| | GSE 55598 | PRAD/ANT (16) | Paired primary PRAD/ANT |
| | GSE 73549 | PRAD/ANT (12) | Paired primary PRAD/ANT |
| | GSE 76938 | PRAD/ANT (52) | Paired primary PRAD/ANT |
| 3: PRAD/Blood | TCGA-PRAD | HCC (315) | Primary PRAD not analyzed |
| Microarray | | | in Layer 2; demographically |
| | | | matched to healthy controls |
| | GSE 84727 | Blood (81) | Healthy control lysed blood |
| | GSE 80417 | Blood (25) | Healthy control lysed blood |
| | GSE 40279 | Blood (89) | Healthy control lysed blood |
| | GSE 72773 | Blood (50) | Healthy control lysed blood |
| | GSE 111629 | Blood (43) | Healthy control lysed blood |
| | GSE 53740 | Blood (27) | Healthy control lysed blood |
| 4: Blood Microarray | GSE 84727 | Blood (224) | Healthy control lysed blood |
| | GSE 80417 | Blood (102) | Healthy control lysed blood |
| | GSE 40279 | Blood (533) | Healthy control lysed blood |
| | GSE 72773 | Blood (222) | Healthy control lysed blood |
| | GSE 111629 | Blood (193) | Healthy control lysed blood |
| | GSE 53740 | Blood (133) | Healthy control lysed blood |

TABLE 2
Diagnostic Odds Ratios (DOR) of
Genes Analyzed by Meta-Analysis

| | | |
|---|---|---|
| Gene | In(DOR) (95% CI) | n |

| | | | |
|---|---|---|---|
| GSTP1 | 2.9 | (2.1, 3.6) | 658 |
| CD44 | 3.3 | (2.0, 4.6) | 69 |
| RARB | 3.1 | (1.2, 5.0) | 127 |
| RASSF1A | 2.1 | (1.1, 3.0) | 317 |
| APC | 1.5 | (0.7, 2.4) | 221 |
| DAPK | 1.8 | (0.5, 3.0) | 146 |
| PYCARD | 2.5 | (0.1, 4.9) | 108 |
| RBP1 | 1.9 | (0.01, 3.7) | 154 |
| CCND2 | 2.9 | (−0.1, 5.9) | 190 |
| ESR2 | 4.1 | (−0.3, 8.5) | 88 |
| CDKN2B | −0.1 | (−1.4, 1.1) | 155 |
| EDNRB | 0.8 | (−1.4, 2.9) | 72 |
| MGMT | −0.7 | (−1.4, 0) | 155 |
| CDKN2A | 0.4 | (−1.8, 2.5) | 184 |
| SFN | 2.7 | (−2.0, 7.4) | 162 |
| ID4 | 0.7 | (−2.3, 3.6) | 124 |
| PTGS2 | 0.2 | (−2.8, 3.2) | 73 |

TABLE 3
Methylation Frequency and AUROC Data for LAMB-PRAD CpGs

| | Gene | PRAD_avg | ANT_avg | Diff | AUC | Blood AUC | Blood_AVG |
|---|---|---|---|---|---|---|---|
| cg16970232 | APC | 0.3755375 | 0.09811316 | 0.2774243 | 0.8561712 | 0.9170673 | 0.06281914 |
| cg14479889 | APC | 0.3490743 | 0.09495405 | 0.2541202 | 0.8485612 | 0.9231847 | 0.05489931 |
| cg22035501 | APC | 0.3195491 | 0.06913557 | 0.2504135 | 0.8316290 | 0.9515344 | 0.03421300 |
| cg14511739 | APC | 0.3100951 | 0.06441692 | 0.2456782 | 0.8300119 | 0.9328395 | 0.02962620 |
| cg23938220 | APC | 0.3529667 | 0.11417396 | 0.2387928 | 0.8607848 | 0.9489242 | 0.02796118 |
| cg11613015 | APC | 0.2880045 | 0.05720559 | 0.2307989 | 0.8263971 | 0.9901335 | 0.01996066 |
| cg00577935 | APC | 0.2869334 | 0.06141711 | 0.2255163 | 0.8387158 | 0.9103250 | 0.04687665 |
| cg08571859 | APC | 0.2976712 | 0.07532352 | 0.2223477 | 0.8412366 | 0.8871353 | 0.06367149 |
| cg08530414 | CD44 | 0.2814812 | 0.10053119 | 0.1809500 | 0.8049465 | 0.8896649 | 0.08140954 |
| cg05907835 | PYCARD | 0.3722229 | 0.17727110 | 0.1949518 | 0.9002616 | 0.9979945 | 0.08258897 |
| cg15468095 | PYCARD | 0.2245480 | 0.06808539 | 0.1564626 | 0.9284185 | 0.9717813 | 0.07326616 |
| cg02499249 | RARB | 0.4473548 | 0.11053802 | 0.3368168 | 0.9178597 | 0.9772638 | 0.07585086 |
| cg26124016 | BARB | 0.3630544 | 0.08706366 | 0.2759907 | 0.8937931 | 0.9347644 | 0.04942677 |
| cg12479047 | RARB | 0.3451416 | 0.07332654 | 0.2718151 | 0.8999287 | 0.9405593 | 0.03902649 |
| cg20899354 | RARB | 0.3389631 | 0.07213856 | 0.2668246 | 0.8976932 | 0.9682439 | 0.02429816 |
| cg18094781 | RARB | 0.3646809 | 0.10321670 | 0.2614642 | 0.8931748 | 0.9269740 | 0.08325233 |
| cg06720425 | RARB | 0.3247715 | 0.09994685 | 0.2248247 | 0.9152913 | 0.9719325 | 0.08118437 |
| cg15229124 | RBP1 | 0.4755218 | 0.16307152 | 0.3124503 | 0.9154340 | 0.9901839 | 0.05650681 |

### REFERENCES

1. Xu et al. (2017) Nature Materials 16:1155-1161.
2. Luo et al. (2020) Science Translational Medicine 12(524) eaax7533.
3. Litwin et al. (2017) JAMA 317(24):2532-2542.
4. American Cancer Society. Cancer Facts & FIGS. 2020. Atlanta: American Cancer Society; 2020.
5. Komura et al. (2018) Int J Urol. 25(3):220-231.
6. Mizokami et al. (2017) Asian J Androl. 19(2):143-148.
7. Gomez-Caamano et al. (2019) Clinical and Translational Oncology 21:420-432.
8. Cornford et al. (2017) European Urology 71:630-642.
9. NCCN Guidelines for Patients Prostate cancer Update. (nccn.org/patients/guidelines/content/PDF/prostate-patient.pdf)
10. Torrecillaa et al. (2015) Reports of Practical Oncology & Radiotherapy 20(4):259-272.
11. NCBI Gene Expression Omnibus (GEO) Accession No. GSE55598 (ncbi.nlm.nih.gov/geo/query/acc.cgi?acc=GSE55598).
12. NCBI Gene Expression Omnibus (GEO) Accession No. GSE112047 (ncbi.nlm.nih.gov/geo/query/acc.cgi?acc=GSE112047).
13. NCBI Gene Expression Omnibus (GEO) Accession No. GSE73549 (ncbi.nlm.nih.gov/geo/query/acc.cgi?acc=GSE73549).
14. NCBI Gene Expression Omnibus (GEO) Accession No. GSE76938 (ncbi.nlm.nih.gov/geo/query/acc.cgi?acc=GSE76938).
15. NCBI Gene Expression Omnibus (GEO) Accession No. GSE108462 (ncbi.nlm.nih.gov/geo/query/acc.cgi?acc=GSE108462).
16. Rescigno et al. (2016) European Urology 70(5):724-731.
17. Nakayama et al. (2016) BMC Urology Volume 16, Article Number: 27.
18. Armstrong et al. (2007) J Clin Oncol 25(25):3965-3970.
19. Fuerea et al. (2016) Eur J Cancer 61:44-51.
20. Petrylak et al. (2006) J Natl Cancer Inst 98(8):516-21.
21. Chen et al. (2014) Biomedicine (Taipei) 4(4): 22.
22. Halgunset et al. (1987) J Steroid Biochem 28(6):731-736.
23. Jutley et al. (1990) Prostate 16(4):299-304.
24. Whelan et al. (1990) Prog Clin Biol Res 357:117-20.
25. Pasquali et al. (1996) J Clin Endocrinol Metab 81(6):2186-91.

1. A method of diagnosing and treating prostate cancer in a patient, the method comprising
measuring frequency of methylation at one or more CpG sites in at least one biomarker gene selected from the group consisting of APC, CD44, PYCARD, RARB, and RBP1 in cell-free DNA (cfDNA) in a patient blood sample, wherein increased frequency of methylation at the one or more CpG sites compared to reference value ranges for the frequency of methylation at the one or more CpG sites in said at least one biomarker gene selected from the group consisting of APC, CD44, PYCARD, RARB, and RBP1 in cfDNA from a control subject indicate that the patient has prostate cancer; and
treating the patient for the prostate cancer, if the patient has a positive diagnosis for prostate cancer based on the frequency of methylation at the one or more CpG sites

2. The method of claim 1, wherein said one or more CpG sites are selected from cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124, and CpG sites located within 200 nucleotides thereof.

3. The method of claim 2, wherein said measuring frequency of methylation comprising measuring the frequency of methylation at the cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124 CpG sites in the cfDNA.

4. The method of claim 1, further comprising calculating a prostate cancer risk score based on the methylation frequency at the CpG sites in the APC, CD44, PYCARD, BARB, and RBP1 genes of the cfDNA using one or more algorithms.

5-9. (canceled)

10. A method of monitoring prostate cancer in a patient, the method comprising:
detecting methylation at one or more CpG sites in one or more genes of circulating free DNA (cfDNA) in a first blood sample obtained from the patient and a second blood sample obtained from the patient, wherein the one or more genes are selected from the group consisting of APC, CD44, PYCARD, RARB, and RBP1, wherein detection of increased frequency of methylation of the CpG sites in the one or more genes selected from the group consisting of APC, CD44, PYCARD, RARB, and RBP1 in the cfDNA of the second blood sample compared to the cfDNA of the first blood sample indicate that the prostate cancer is progressing, and detection of decreased frequency of methylation of the CpG sites in the one or more genes selected from the group consisting of APC, CD44, PYCARD, RARE, and RBP1 in the cfDNA of the second blood sample compared to the cfDNA of the first blood sample indicate that the prostate cancer is not progressing.

11-19. (canceled)

20. A method of monitoring for a recurrence of prostate cancer in a patient, the method comprising:
a) detecting methylation at one or more CpG sites of one or more biomarker genes in cfDNA from a first circulating free DNA (cfDNA sample) obtained from the patient after treatment for a previous occurrence of prostate cancer at a first time point when the patient is characterized as cancer-free from imaging or other diagnostic modalities, wherein the one or more biomarker genes are selected from APC, CD44, PYCARD, RARB, and RBP1;
b) detecting methylation at the one or more CpG sites of the one or more biomarker genes in cfDNA from a second cfDNA sample obtained from the patient at a second time point during a period of monitoring for the recurrence, wherein the one or more biomarker genes are selected from APC, CD44, PYCARD, RARB, and RBP1, wherein increased frequency of methylation at the one or more CpG sites of the one or more biomarker genes selected from APC, CD44, PYCARD, RARB, and RBP1 in the cfDNA of the second cfDNA sample compared to the cfDNA of the first cfDNA sample indicates that the prostate cancer has recurred; and
c) repeating step b subsequently during the period of monitoring for the recurrence.

21. The method of claim 20, further comprising treating the patient for the recurrence of the prostate cancer, if the patient has a positive diagnosis for the recurrence of the prostate cancer based on the levels of methylation of the one or more CpG sites.

22. The method of claim 20, wherein said treating the patient for the recurrence of prostate cancer comprises surgical resection, prostatectomy, external beam radiation therapy, particle therapy, brachytherapy, cryosurgery, high-intensity focused ultrasound, androgen deprivation therapy, chemotherapy, targeted therapy, or immunotherapy, or a combination thereof.

23. The method of claim 20, wherein the one or more CpG sites are selected from cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124, and CpG sites located within 200 nucleotides thereof.

24. The method of claim 23, wherein said detecting methylation comprises measuring frequency of methylation at the cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124 CpG sites in the cfDNA.

25-27. (canceled)

28. A method for selecting an individual with prostate cancer for treatment with abiraterone acetate (17-(3-pyridinyl)androsta-5,16-dien-3β-ol acetate) and treating the individual, the method comprising:
a) measuring frequency of methylation at one or more CpG sites in at least one biomarker gene selected from the group consisting of RARB and RBP1 in cell-free DNA (cfDNA) in a blood sample obtained from the individual, wherein increased frequency of methylation at the one or more CpG sites compared to reference value ranges for the frequency of methylation at the one or more CpG sites in said at least one biomarker gene selected from the group consisting of RARB and RBP1 in cfDNA from a control subject indicate that the individual will benefit from treatment with the abiraterone acetate; and
b) administering a therapeutically effective amount of the abiraterone acetate to the individual, if the frequency of methylation at the one or more CpG sites in said at least one biomarker gene selected from the group consisting of RARB and RBP1 indicates that the individual will benefit from treatment with the abiraterone acetate.

29. The method of claim 28, wherein said one or more CpG sites are selected from cg02499249, cg26124016, cg12479047, cg20899354, cg18094781, cg06720425, and cg15229124.

30. The method of claim 29, wherein said measuring frequency of methylation comprises measuring the frequency of methylation at the cg02499249, cg26124016, cg12479047, cg20899354, cg18094781, cg06720425, and cg15229124 CpG sites in the cfDNA.

31. (canceled)

32. A kit comprising agents for detecting methylation of one or more CpG sites in APC, CD44, PYCARD, RARB, and RBP1 genes in cfDNA.

33. The kit of claim 32, wherein said one or more CpG sites are selected from cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124, and CpG sites located within 200 nucleotides thereof.

34. The kit of claim 33, wherein said CpG sites comprise cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124.

35-38. (canceled)

39. A cell-free DNA hypermethylated at one or more CpG sites in at least one biomarker gene selected from APC, CD44, PYCARD, RARB, and RBP1 for use in diagnosing prostate cancer, detecting recurrence of prostate cancer, or monitoring treatment of prostate cancer.

40. The cell-free DNA of claim 39, wherein said one or more CpG sites are selected from cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124, and CpG sites located within 200 nucleotides thereof.

41. An in vitro method of diagnosing prostate cancer in a patient, the method comprising:
measuring frequency of methylation at one or more CpG sites in at least one biomarker gene selected from the group consisting of APC, CD44, PYCARD, RARB, and RBP1 in cell-free DNA (cfDNA) in a blood sample obtained from the patient, wherein increased frequency of methylation at the one or more CpG sites compared to reference value ranges for the frequency of methylation at the one or more CpG sites in said at least one biomarker gene selected from the group consisting of APC, CD44, PYCARD, RARB, and RBP1 in cfDNA from a control subject indicate that the patient has prostate cancer.

42. The in vitro method of claim 41, wherein said one or more CpG sites are selected from cg16970232, cg14479889, cg22035501, cg14511739, cg23938220, cg11613015, cg00577935, cg08571859, cg08530414, cg05907835, cg15468095, cg02499249, cg26124016, cg12479047, cg20899354, cg18094781, cg06720425, and cg15229124, and CpG sites located within 200 nucleotides thereof.

43. (canceled)
